(19) United States
(12) Patent Application Publication
(10) Pub. No.: US 2026/0146233 A1
(43) Pub. Date: May 28, 2026

(54) HIF-1a STABILIZATION FOR TISSUE REGENERATION

(71) Applicant: The University of Hong Kong, Hong Kong (CN)

(72) Inventors: Waruna Lakmal Dissanayaka, Hong Kong (CN); Hong Wang, Hong Kong (CN); Mohamad Koohi-Moghadam, Hong Kong (CN)

(21) Appl. No.: 19/395,710

(22) Filed: Nov. 20, 2025

Related U.S. Application Data
(60) Provisional application No. 63/724,114, filed on Nov. 22, 2024.

(51) Int. Cl. C12N 5/077; A61K 35/32

(52) U.S. Cl. CPC C12N 5/0654; A61K 35/32; C12N 2501/165

(57) ABSTRACT

Ensuring cell survival and promoting rapid vascularization after transplantation is an important for achieving successful tissue regeneration. Disclosed herein are methods designed to enhance cell survival and stimulate neovascularization and/or vascularized tissue regeneration after transplantation in a subject. The disclosed approach involves using a compound to pre-treat cells, stabilizing or activating HIF-1α under normal oxygen conditions, thereby increasing the therapeutic potential of the treated cells and triggering an angiogenic/vasculogenic response, particularly for dental pulp regeneration.

### CROSS-REFERENCE TO RELATED APPLICATIONS

This application claims benefit of and priority to U.S. Provisional Application No. 63/724,114, filed Nov. 22, 2024, which is specifically incorporated by reference herein in its entirety.

### FIELD OF THE INVENTION

The disclosed invention is generally in the field of tissue regeneration and specifically in the area of using compounds to stabilize HIF-1α for dental pulp regeneration.

### BACKGROUND OF THE INVENTION

Due to the unique features of the craniofacial structures, the defects of the tissues caused by trauma, tumors, inflammation, and other disorders can seriously influence the basic functions and aesthetics of the patients^{[1]}. Recently, cell-based tissue engineering strategies, such as dental pulp regeneration, periodontal tissue regeneration, and bone regeneration, have been widely studied as a treatment option in craniofacial diseases as well as some systemic diseases, in which neovascularization is one of the important factors that decides the outcome^{[2]}. Despite these developments, the clinical success of such regenerative strategies remains limited because transplanted cells must survive, integrate, and rapidly form microvascular networks within host tissues that are often hypoxic and nutrient-deprived. For example, it has been reported that therapeutics based on cell transplantation may have poor efficacy due to the changes in oxygen concentration between in vitro and in vivo conditions^{[3,4]}. Following implantation into ischemic or poorly vascularized tissues, only 10-60% of cells survive, as shown using ischemic models^{[5]}. Such low survival rates are largely attributed to the abrupt transition from well-oxygenated culture conditions to severely hypoxic tissue microenvironments, leading to oxidative stress, mitochondrial dysfunction, and apoptosis of transplanted cells before sufficient vascular ingrowth occurs. The rates could well be higher in cell-based pulp-tissue engineering due to the anatomical limitation of the tooth, which receives blood supply from a small apical opening^{[3,4]}. In addition, the confined nature of the pulp cavity limits the diffusion of oxygen and nutrients, making the establishment of a functional microcirculation even more important. Therefore, ensuring sustained cell viability and inducing rapid neovascularization after transplantation remain major obstacles, as inadequate vascular integration leads to early cell loss and incomplete tissue regeneration. This represents an urgent problem that needs to be addressed in dental tissue regeneration strategies.

Emerging evidence suggested that hypoxia-inducible factor-1 alpha (HIF-1α) stabilization or activation induced by hypoxia preconditioning could be an endogenous regulatory means to reduce hypoxia-induced damage and a potent way to enhance cell survival^{[6,7]}. HIF-1α serves as a master transcriptional regulator of the cellular response to low oxygen, promoting the expression of pro-survival and angiogenic factors such as vascular endothelial growth factor (VEGF), erythropoietin, and glucose transporters. Stabilization of HIF-1α prior to implantation can facilitate pre-adaptation of stem cells to hypoxic stress.

Stem cells from Human Exfoliated Deciduous teeth (SHED) have been identified as a population of highly proliferative and clonogenic dental stem cells that are able to induce bone formation, dentin regeneration, neuroregeneration, and angiogenesis Due to the superior stem cell properties and the non-invasive nature of stem cells from exfoliated teeth, SHED have become important cells in stem cell-based autologous transplantation and tissue engineering^{[11,12]}. However, translating the benefits of hypoxic preconditioning into practical and clinically safe protocols remains a major challenge. Current genetic approaches, such as lentiviral-mediated knockdown of prolyl hydroxylase domain (PHD) enzymes, can effectively stabilize HIF-1α but pose biosafety risks, regulatory hurdles, and potential insertional mutagenesis, precluding their clinical use. Similarly, chemical stabilizers of HIF-1α, such as cobalt chloride (CoCl₂) and deferoxamine (DFO), act by chelating Fe²⁺ and inhibiting PHD activity but are often cytotoxic, induce nonspecific stress responses, and fail to accurately replicate the physiological hypoxic signaling in dental stem cells.

More interestingly, it has been shown that HIF-1α expression in SHED is significantly stabilized in normoxia following the knockdown of PHD, which in turn improves the angio-/vasculogenic properties and post-implantation cell survival^{[13-15]}. Therefore, it is important to identify and characterize compound(s) and dosage forms for applying the hypoxic preconditioning phenomenon in SHED. There is a need for maintenance of cell survival and rapid vascularization after cellular transplantation. In particular, there remains a need for small-molecule agents that can safely and reversibly induce HIF-1α stabilization, increase the angiogenic potential of SHED, and maintain their viability in the hypoxic and nutrient-restricted environment following transplantation.

It is therefore an object of the present invention to identify and use compounds to stabilize or activate HIF-1α, which contributes to the success of cell-based tissue regeneration.

It is an object of the invention to provide compositions of preconditioned dental mesenchymal stem cells with increased regenerative potential.

It is another object of the invention to provide methods for increasing the survival and regenerative ability of stem cells after transplantation.

It is yet another object of the invention to provide methods for treating dental or craniofacial conditions using the preconditioned dental mesenchymal stem cells.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification, the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### BRIEF SUMMARY OF THE INVENTION

Disclosed are treated stem cells and methods for enhancing cell survival and promoting neovascularization and/or vascularized tissue regeneration following transplantation in a subject. In some forms, the stem cells are treated ex vivo with a compound in an amount effective to stabilize, activate, or express hypoxia-inducible factor-1 alpha (HIF-1α) in the stem cells under normoxic conditions.

In some forms, the stem cells are obtained from human exfoliated deciduous teeth (SHED). In some forms, the compound is a Bcl-2 family inhibitor. In some forms, the Bcl-2 family inhibitor is selected from a group consisting of a small molecule, a peptide, an antibody, a nucleic acid, or a combination thereof. In some forms, the small molecule is TW-37, ABT-737, ABT-263, BM-1197, S44563, AZD4320, ABT-199, or S55746. In some forms, the small molecule is TW-37.

In some forms, the effective amount of the compound ranges from about 1 μM to 20 μM. In some forms, the compound stabilizes, activates, or expresses HIF-1α through the modulation of adenylate kinase 4 (AK4). In some forms, the stabilization, activation, or expression of HIF-1α results in maintenance of ATP homeostasis. In some forms, the modulation of adenylate kinase 4 (AK4) maintains ATP homeostasis. In some forms, the compound results in maintenance of ATP homeostasis.

Generally, the method involves administering the treated stem cells to a subject. In some forms, the treated stem cells enhance the survival of the stem cells in the subject. In some forms, the treated stem cells promote neovascularization in the subject. In some forms, the treated stem cells promote regeneration of vascularized tissue in the subject.

In some forms, the stabilization or the activation of HIF-1α induces angiogenic and/or vasculogenic responses. In some forms, the angiogenic and/or vasculogenic responses are mediated through the expression of an endothelial growth factor. In some forms, the endothelial growth factor is platelet-derived growth factor (PDGF), fibroblast growth factor (FGF), or angiogenin. In some forms, the endothelial growth factor is vascular endothelial growth factor (VEGF).

In some forms, the treated stem cells are administered into the subject by intravenous (IV) injection, intramuscular (IM) injection, intra-articular injection, intrathecal injection, direct tissue injection, intra-arterial injection, subcutaneous injection, topical application, scaffold-based transplantation, or a combination thereof.

In some forms, the administered treated stem cells promote neovascularization or vascular sprouting of endothelial cells through HIF-la-induced VEGF expression. In some forms, the stabilization, activation, or expression of HIF-1α under normoxia in SHED enhances the therapeutic potential of the stem cells in SHED for dental pulp regeneration through augmentation of angiogenic properties and post-transplantation cell survival.

### BRIEF DESCRIPTION OF THE DRAWINGS

FIGS. 1A-1F. TW-37 induced HIF-1α stabilization. Western blot results showed that protein expression levels of HIF-1α were significantly increased by TW-37 in SHED under normoxia in a dose-dependent manner at 6 h (FIGS. 1A-1B), 12 h (FIGS. 1C-1D) and 24 h (FIGS. 1E-1F), respectively. *P<0.05, **P<0.01, ***P<0.001.

FIGS. 2A-2B. TW-37 inhibited SHED viability after 48 hours. CCK-8 assay showed no statistically significant difference in cell viability from 0 to 10 μM concentrations of TW-37, but indicated a statistically significant inhibition of SHED viability at 20 μM after 24 h (FIG. 2A). Almost all the concentrations showed significant reduction in cell viability of SHED following the treatment for 48 and 72 h (FIG. 2A). Interestingly, from the results of the Live & Dead assay (FIG. 2B), almost no distinct dead cells were detected at 24 h when comparing the TW-37-treated groups with the control group, which indicated that TW-37 inhibited SHED proliferation but did not induce cell death. *P<0.05, ***P<0.001.

FIGS. 3A-3E. TW-37 promoted the secretion of VEGF. Secretory VEGF levels at 6 h (FIG. 3A), 12 h (FIG. 3B) and 24 h (FIG. 3C) in SHED with the treatment of TW-37 were increased markedly in a dose-dependent manner as detected by ELISA. Then, after the stimulation of TW-37 on SHED for 12 h or 24 h, the supernatant of the cells was removed and fresh medium was added to the wells to continue to culture the cells for another 24 h. The ELISA results indicated that SHED could still secrete a high level of VEGF, significantly in 10 μM and 20 μM groups, after TW-37 being removed (FIGS. 3D-3E). The above results suggested that TW-37 could promote the secretion of VEGF both directly and indirectly. *P<0.05, **P<0.01 and ***P<0.001.

FIGS. 4A-4E. TW-37-induced SHED enhanced angiogenesis of HUVECs. To assess the influence of TW-37-induced SHED on angiogenesis, conditioned medium was collected after SHED was treated with 10 μM TW-37 for 24 h. In vitro Matrigel tube formation assay showed that the numbers of nodes were significantly increased at 6 h and 12 h, but with no significant increase after 24 h, which indicates that TW-37 could enhance the angiogenesis at the early stage but did not stabilize tube formation in the long term (FIGS. 4A-4B). The sprouting assay revealed that the conditioned medium from TW-37-induced SHED promoted the sprouting of HUVECs in a 3D spheroid model (FIGS. 4C-4E). *P<0.05 and ***P<0.001.

FIGS. 5A-5F. TW-37 did not function as a PHD inhibitor in SHED. Western blot results showed that, with treatment of SHED with TW-37, HIF-1α expression first increased and then decreased, with the highest HIF-1α expression at 3 h (FIGS. 5A-5B). Interestingly, when HIF-1α was stabilized by TW-37, the expression of PHD was upregulated, which may indicate that the upregulation of HIF-1α induces PHD (FIGS. 5C-5E). Then the function of TW-37 on Fe²⁺ was detected by ferrozine assay (FIG. 5F). The results suggested that TW-37 could not act as a Fe²⁺ chelator in SHED. *P<0.05, **P<0.01 and ***P<0.001.

FIGS. 6A-6F. RNA sequencing. RNA sequencing was done to further explore the function and mechanism of TW-37 on SHED. The sequencing results demonstrated that there are 803 different expressed genes, including 479 upregulated genes and 324 downregulated genes at 3 h (FIGS. 6A-6B). The analysis of the statistical pathway enrichment of all differentially expressed genes showed that the HIF-1α signaling pathway is the most involved pathway apart from the pathways in cancer (FIG. 6C). When it comes to the upregulated genes, it can be seen that the upregulated genes had a higher relation with the HIF-1α signaling pathway (FIG. 6D). FIGS. 6E-6F: Different expressed genes between the TW-37 group and the hypoxia group (the hypoxia chamber) were calculated to detect how TW-37 mimics hypoxia. The result showed there are 367 different expressed genes, including 154 upregulated genes and 213 downregulated genes.

FIGS. 7A-7D. TW-37 promoted HIF-1α stabilization through regulating AK4. The results from the western blot showed that HIF-1α and AK4 were both enhanced by TW-37 (FIG. 7A). Then, AK4 siRNA was used to further explore the interaction of HIF-la and AK4. Si-2 was selected for the following experiments from the detection of the efficiency of AK4 siRNA (FIG. 7B). The results showed that HIF-1α was inhibited when AK4 was knocked down with the treatment of TW-37 at 1.5 h and 3 h (FIGS. 7C-7D).

FIG. 8. Schematic illustration of the screening of TW-37. RNA-Seq data of PHD₂^{KD}-SHED were processed to extract up-/down-regulated genes. Transcript profile of PHD₂^{KD}-SHED was compared with the unique signature of a drug treatment in the Connectivity Map (CMap) database and TW-37 was selected.

FIGS. 9A-9F. Effect of CoCl₂ and DFO on SHED. CoCl₂ (FIGS. 9A-9B) and DFO (FIGS. 9C-9D) induced HIF-1α stabilization at both 6 h (FIGS. 9A, 9C) and 24 h (FIGS. 9B, 9D) in SHED. CCK-8 assay showed that CoCl₂ (FIG. 9E) and DFO (FIG. 9F) had significant cytotoxicity on SHED. *P<0.05, **P<0.01 and ***P<0.001.

### DETAILED DESCRIPTION OF THE INVENTION

The disclosed method can be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### A. Definitions

“Normoxic conditions”, as used herein, generally refer to standard atmospheric oxygen levels present during routine cell culture, such as about 20-21% oxygen (O₂) and 5% carbon dioxide (CO₂) at 37° C. in a humidified incubator. Under these conditions, cells are exposed to oxygen concentrations similar to ambient air rather than the reduced oxygen levels found in hypoxic tissues (e.g., 1-5% O₂).

“Hypoxic conditions,” as used herein, generally refer to oxygen levels lower than those present under standard atmospheric or normoxic conditions, e.g., below about 5% oxygen (O₂), such as within the range of 0.5% to 3% O₂, depending on the desired degree of hypoxic stress. Such conditions approximate the oxygen-deprived microenvironments encountered in vivo within ischemic, inflamed, or poorly vascularized tissues.

“Hypoxia-mimetic agent,” as used herein, generally refers to a compound or molecule capable of inducing cellular responses similar to those triggered by low-oxygen (hypoxic) conditions.

As used herein, the term “dosage regime” refers to drug administration regarding formulation, route of administration, drug dose, dosing interval and treatment duration.

As used herein, the terms “individual”, “host”, “subject”, and “patient” are used interchangeably, and refer to a mammal, including, but not limited to, primates, for example, human beings, as well as rodents, such as mice and rats, and other laboratory animals.

As used herein, the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered. The effect of the effective amount can be relative to a control. Such controls are known in the art and discussed herein, and can be, for example, the condition of the subject prior to or in the absence of administration of the drug, or drug combination, or in the case of drug combinations, the effect of the combination can be compared to the effect of administration of only one of the drugs.

Use of the term “about” is intended to describe values either above or below the stated value in a range of approx. +/−10%; in other forms, the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

The use of the terms “a,” “an,” “the,” and similar referents in the context of describing the present disclosure (especially in the context of the claims) is to be construed to cover both the singular and the plural, unless otherwise indicated herein or clearly contradicted by context.

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist.” One that decreases, or prevents, a known activity is an “antagonist.”

The term “inhibit” or other forms of the word, such as “inhibiting” or “inhibition”, means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” can also mean to hinder or restrain the synthesis, expression or function of a protein relative to a standard or control. Inhibition can include, but is not limited to, the complete ablation of the activity, response, condition, or disease. “Inhibits” can also include, for example, a 10% reduction in the activity, response, condition, disease, or other biological parameter as compared to the native or control level. Thus, the reduction can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels. For example, “inhibits expression” means hindering, interfering with or restraining the expression and/or activity of the gene/gene product pathway relative to a standard or a control.

The term “monitoring” as used herein refers to any method in the art by which an activity can be measured.

The term “providing” as used herein refers to any means of adding a compound or molecule to something known in the art. Examples of providing can include the use of pipettes, pipettemen, syringes, needles, tubing, guns, etc. This can be manual or automated. It can include transfection by any means or any other means of providing nucleic acids to dishes, cells, tissue, cell-free systems and can be in vitro or in vivo.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a caregiver's expertise, but that include the knowledge that the subject is ill, or will be ill, as a result of a condition that is treatable by the disclosed compounds.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward the removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

A cell can be in vitro. Alternatively, a cell can be in vivo and can be found in a subject. A “cell” can be a cell from any organism including, but not limited to, a bacterium.

In one aspect, the compounds described herein can be administered to a subject comprising a human or an animal, including, but not limited to, a mouse, dog, cat, horse, bovine, or ovine, and the like, that is in need of alleviation or amelioration from a recognized medical condition.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed, that while specific reference of each various individual and collective combinations and permutations of these compounds may not be explicitly disclosed, each is specifically contemplated and described herein. For example, if a ligand is disclosed and discussed, and a number of modifications that can be made to a number of molecules, including the ligand, are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F is specifically contemplated and should be considered disclosed from the disclosure of A, B, and C; D, E, and F; and the example combination A-D. Likewise, any subset or combination of these is also specifically contemplated and disclosed. Thus, for example, the sub-group of A-E, B-F, and C-E is specifically contemplated and should be considered disclosed from the disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application, including, but not limited to, steps in methods of making and using the disclosed compositions. Thus, if there are a variety of additional steps that can be performed, it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the invention.

Recitation of ranges of values herein is merely intended to serve as a shorthand method of referring individually to each separate value falling within the range, unless otherwise indicated herein, and each separate value is incorporated into the specification as if it were individually recited herein.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents form part of the common general knowledge in the art.

### B. Compositions of Pre-Conditioned Dental Mesenchymal Stem Cells

Dental and craniofacial tissue regeneration is often limited by the poor survival of transplanted cells in oxygen-deprived environments and by the delayed formation of new blood vessels at the implantation site. In particular, dental mesenchymal stem cells (MSCs) rapidly lose viability when transferred from nutrient-rich culture conditions to the confined and hypoxic environment of the tooth or surrounding tissues, resulting in insufficient vascularization and incomplete tissue repair.

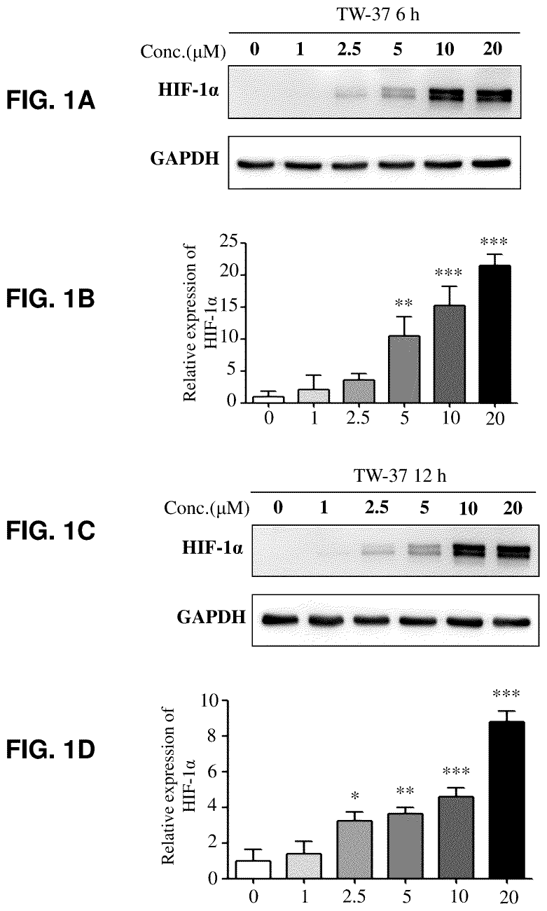

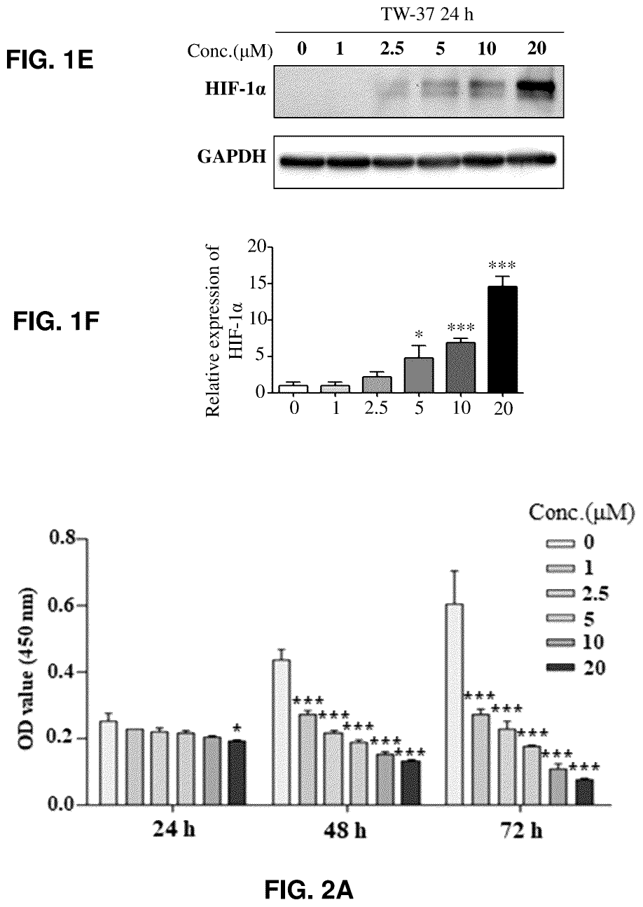

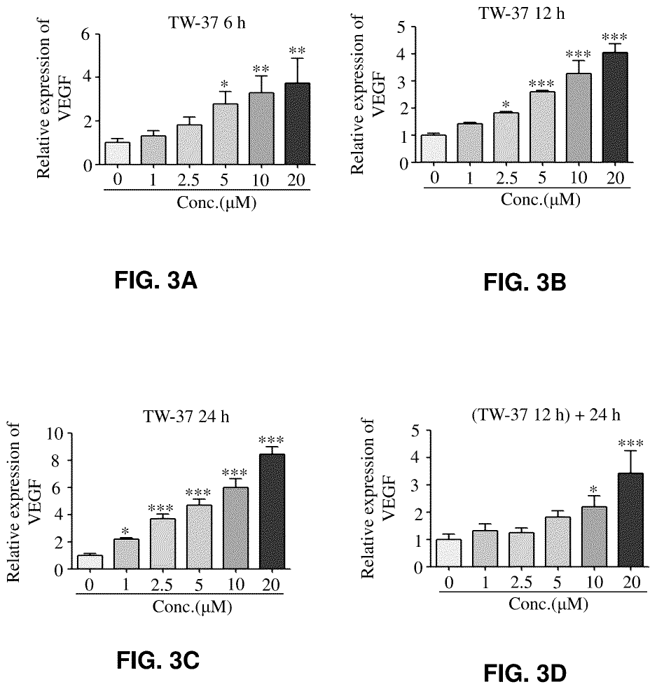

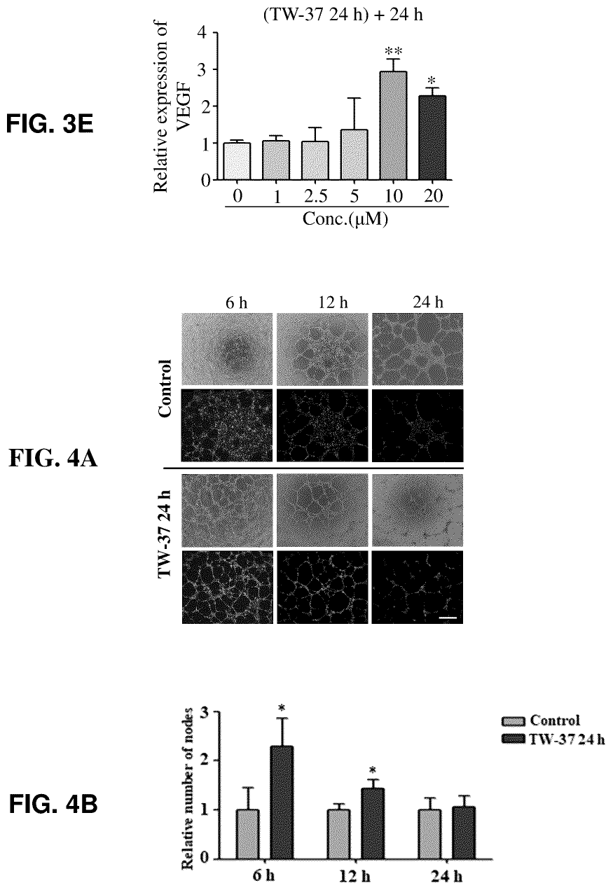

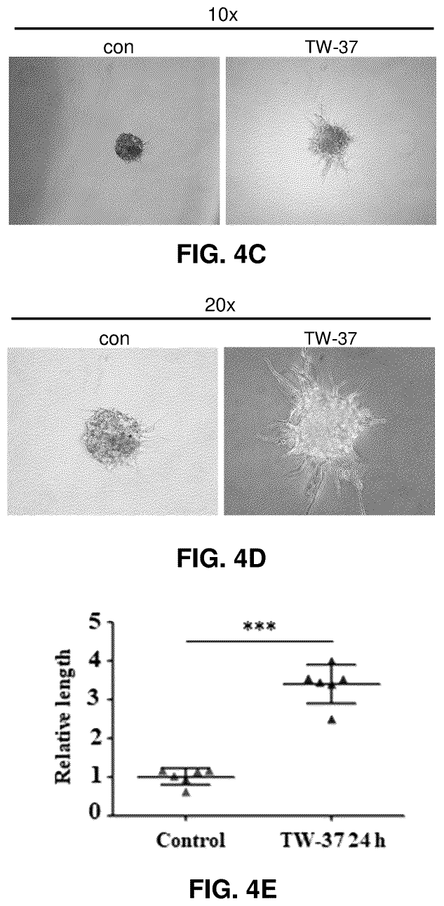

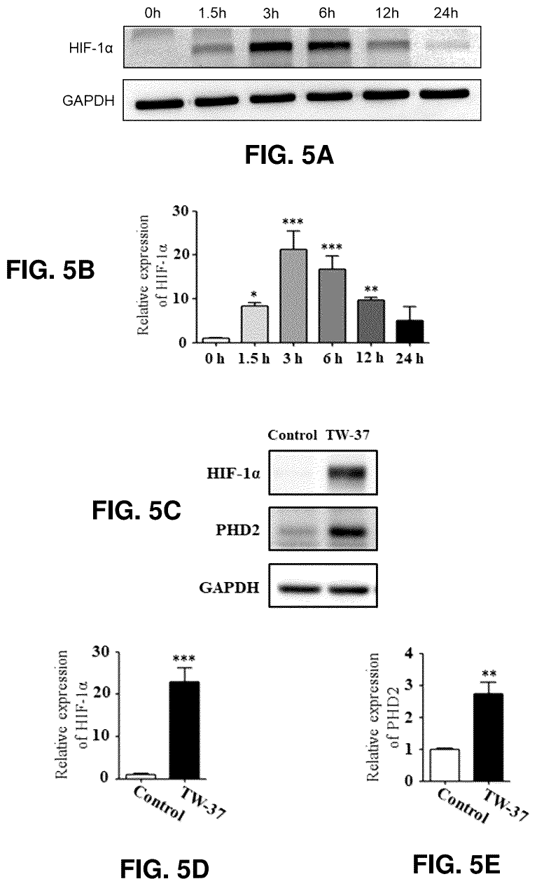

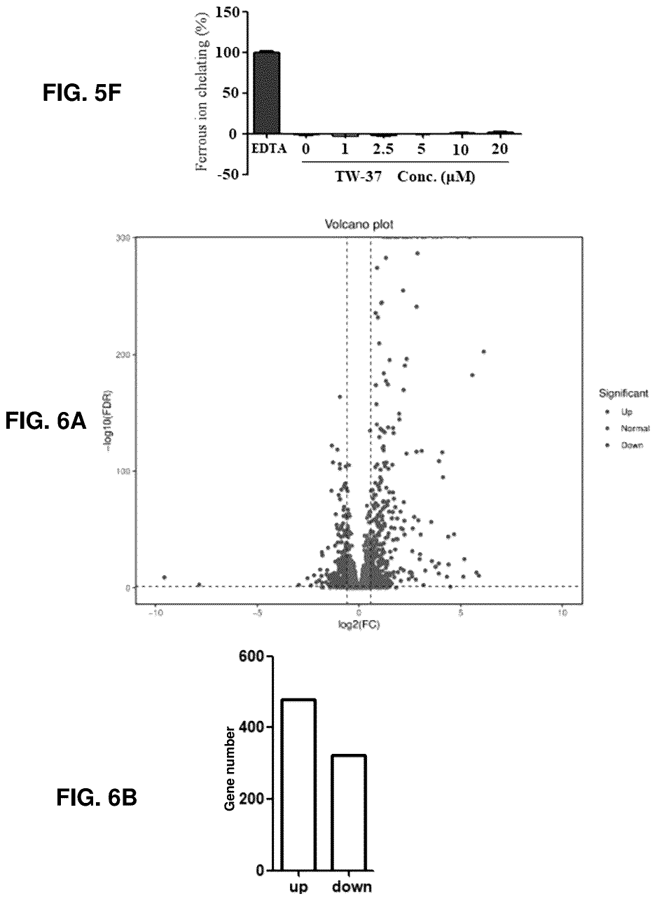

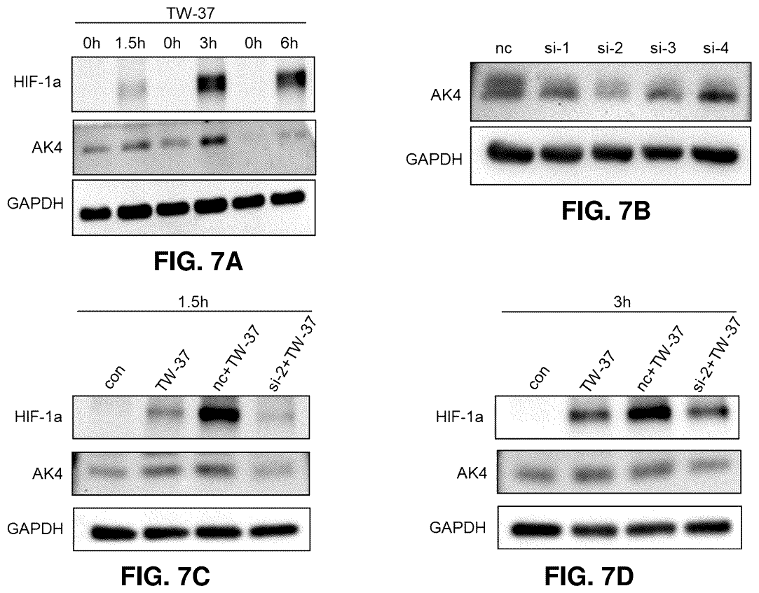

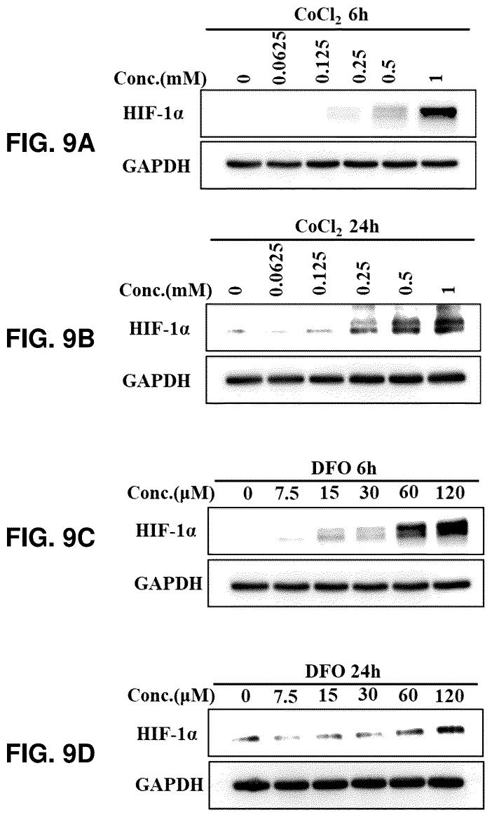

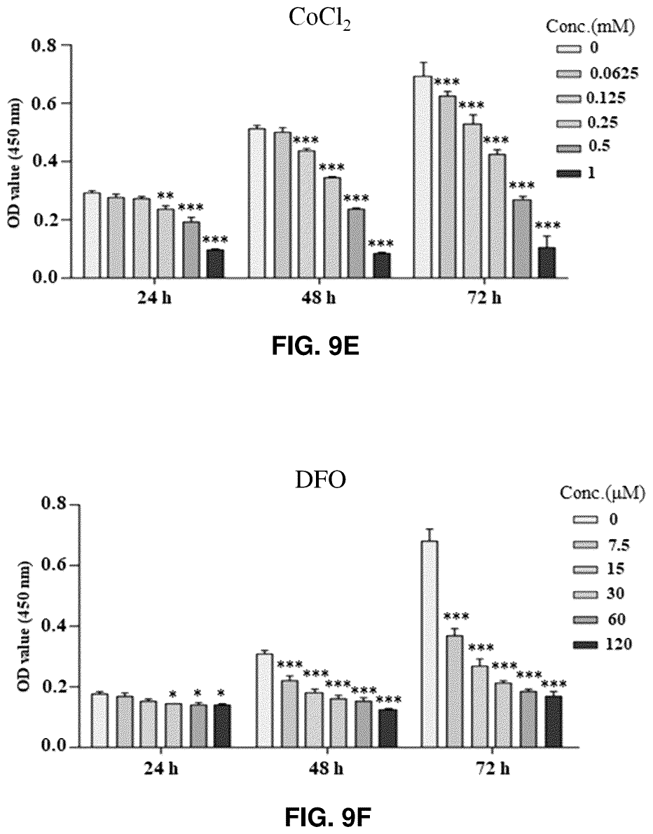

To address these challenges, a preconditioning strategy was developed, in which dental MSCs are exposed ex vivo to the exemplary small-molecule agent, TW-37, under conditions to mimic hypoxia and increase the resistance of the dental MSCs to oxygen deprivation. This preconditioning step activates endogenous protective pathways and increases the secretion of angiogenic factors that support vascularization and dental stem cell integration. As demonstrated in the non-limiting Examples herein, treatment of stem cells from human exfoliated deciduous teeth (SHED) with the exemplary small molecule TW-37, led to stabilization of HIF-1α, upregulation of adenylate kinase 4 (AK4), and a marked increase in vascular endothelial growth factor (VEGF) secretion (FIGS. 1A-IF, FIGS. 3A-3E, and FIGS. 7A-7D, respectively). The pre-conditioned SHED cells exhibited increased angiogenic activity in vitro, as shown by increased endothelial sprouting and tube formation (FIGS. 4A-4E). The observed effects were not dependent on the classical hypoxia-mimetic mechanisms of conventional HIF-1α stabilizers (e.g., cobalt chloride (CoCl2), or deferoxamine (DFO), such as iron chelation or direct inhibition of prolyl hydroxylase enzymes (FIGS. 5C-5F, 9A-9F).

Therefore, compositions of pre-conditioned dental mesenchymal stem cells (MSCs) are provided (herein referred to as “pre-conditioned dental MSCs” or generally “stem cells”). The compositions are particularly suitable for use in regenerative therapies, including but not limited to dental pulp regeneration, periodontal tissue repair, and craniofacial soft- or hard-tissue reconstruction, where increased cell survival and rapid neovascularization are important for successful tissue regeneration.

The compositions include a population of dental mesenchymal stem cells (e.g., SHED cells) that have been pre-treated with a preconditioning agent (e.g., TW-37) capable of stabilizing hypoxia-inducible factor-1 alpha (HIF-1α) and increasing cellular resistance to hypoxic or stress conditions. The compositions are formulated in a biocompatible carrier or medium suitable for maintaining cell viability and facilitating administration to a target tissue site (e.g., dental pulp, periodontal tissue, alveolar bone, or other craniofacial soft or hard tissues requiring regeneration). In some forms, the carriers include, without limitation, buffers such as saline and appropriate culture media. In other forms, the carriers include biocompatible hydrogels and scaffolds composed of materials such as collagen, fibrin, hyaluronic acid, alginate, or gelatin.

The compositions of pre-conditioned dental MSCs (e.g., pre-treated SHED cells), can be provided as an injectable cell suspension, cryopreserved preparation, or scaffold-embedded constructs for use in dental of craniofacial regenerative applications.

### 1. Dental Mesenchymal Stem Cells

Dental mesenchymal stem cells (dental MSCs) are a readily accessible and minimally invasive source of postnatal stem cells derived from dental tissues. These cells possess self-renewal capacity and multilineage differentiation potential, permitting them to give rise, under appropriate regenerative conditions, to odontogenic, osteogenic, and neurogenic lineages. In addition to their differentiation potential, dental MSCs exhibit strong angiogenic and trophic properties that promote vascularization, tissue repair, and functional regeneration (e.g., dental pulp regeneration).

The dental MSCs include but are not limited to dental pulp stem cells (DPSCs), stem cells from human exfoliated deciduous teeth (SHED), periodontal ligament stem cells (PDLSCs), stem cells from the apical papilla (SCAP), and dental follicle stem cells (DFSCs). These cells can be characterized by their adherence in standard culture conditions, expression of mesenchymal surface markers (e.g., CD73, CD90, CD105), and lack of hematopoietic markers (e.g., CD34, CD45).

In one exemplary form, the dental MSCs are human exfoliated deciduous teeth (SHED) cells. SHED cells are a more restricted and clonogenic population of human dental pulp stem cells. These SHED cells exhibit rapid proliferation in vitro, a high colony-forming efficiency, and expression of mesenchymal stem cell markers such as STRO-1, CD146, CD73, CD90, and CD105, while lacking hematopoietic markers such as CD34 and CD45 (Miura et al., 2003; Gronthos et al., 2000; 2002).

In culture, SHED cells can exhibit a fibroblast-like morphology and grow as adherent monolayers under culture conditions e.g., α-minimum essential medium (α-MEM) supplemented with fetal bovine serum and antibiotics at about 37° C. and about 5% CO₂ (see FIGS. 4C-4G). In some forms, the SHED cells demonstrate robust metabolic activity and maintain high viability when assessed by live/dead fluorescence staining, with negligible spontaneous cytotoxicity under normal culture conditions. In some forms, the SHED cells proliferate in a time-dependent manner, as can be confirmed by colorimetric viability assays, and retain a stable phenotype across multiple passages.

In some forms, the SHED cells are capable of differentiating into odontoblast-like, osteoblast-like, and neuron-like cells. In some forms, the SHED cells can form dentin- and bone-like mineralized tissues in vivo following transplantation into a subject. For example, it has been observed that culturing SHED cells and related dental pulp stem cells under lineage-specific differentiation conditions resulted in the generation of specialized cell types (e.g., odontoblast-like cells), as well as the production of mineralized matrices resembling dentin and bone following transplantation into immunocompromised mice (About et al., 2000, Experimental Cell Research, 258(1):33-41; Gronthos et al., 2002, Journal of Dental Research, 82(8): 531-535; Miura et al., 2003, Proceedings of the National Academy of Sciences of the United States of America. 2003; 100(10): 5807-5812). Additionally, as demonstrated in the Examples, undifferentiated SHED cells secrete pro-angiogenic factors, such as vascular endothelial growth factor (VEGF), which promote endothelial sprouting and tube formation, demonstrating a strong paracrine role in supporting angiogenesis and vascularized tissue regeneration.

The dental MSCs can be isolated from dental tissues of deciduous or permanent teeth, including but not limited to the dental pulp, periodontal ligament, dental follicle, and apical papilla. In preferred forms, the dental MSCs are isolated from dental pulp tissues, which are the soft connective tissues located in the central chamber of the tooth. The dental pulp contains a rich vascular and neural network and houses stem cells capable of differentiating into odontoblast-like cells that contribute to dentin formation and pulp regeneration.

In some forms, the dental MSCs are isolated from periodontal ligament tissues, which are the fibrous connective tissues anchoring the tooth root to the alveolar bone. Periodontal ligament stem cells exhibit strong osteogenic and cementogenic potential and are useful for periodontal tissue regeneration and repair of alveolar bone defects. In some forms, the dental MSCs are derived from the dental follicle, a loose ectomesenchymal tissue that surrounds the developing tooth germ. Dental follicle stem cells are precursors of cementoblasts, osteoblasts, and periodontal ligament fibroblasts and thus serve as an important source for forming the supporting structures of the tooth. In some forms, the dental MSCs are isolated from the apical papilla, which is the soft tissue located at the apex of an immature permanent tooth root. Stem cells from the apical papilla are highly proliferative and exhibit both odontogenic and osteogenic potential, making them particularly useful for root maturation and pulp-dentin complex regeneration.

The dental MSCs can be obtained from intact, extracted, or exfoliated teeth, which are enzymatically or mechanically processed to release the stromal cell fraction containing the stem cell population. For example, the dental MSCs can be isolated from the dental pulp of naturally exfoliated deciduous teeth by carefully sectioning the tooth to expose the pulp chamber, followed by enzymatic digestion using a collagenase/dispase solution to dissociate the pulp tissue. The resulting cell suspension can then be filtered and plated in a suitable culture medium (e.g., α-minimum essential medium supplemented with fetal bovine serum and antibiotics) under standard incubation conditions to allow adherence and expansion of the mesenchymal stem cell population. The isolated cells, referred to as SHED cells, generally exhibit fibroblast-like morphology, express mesenchymal stem cell markers, and display strong proliferative and differentiation capacity.

Techniques for isolating and expanding dental MSCs are known in the art, e.g., as described in U.S. Pat. No. 6,767,740 to Kerkis et al., and U.S. Patent Application Publication No. 2004/0058442 to Shi et al., the contents of which are incorporated herein by reference in their entireties. For example, studies on dental pulp-derived stem cells from humans and rats have established standard approaches for isolating and characterizing cells with osteogenic and odontogenic potential. Early investigations demonstrated that pulp-derived cells are capable of differentiating in vivo into bone and dentin, confirming their regenerative capacity in mineralized tissues (Yamamura, 1985, Journal of Dental Research, 64(Spec. No.): 530-540; Caplan, 1991, Journal of Orthopaedic Research, 9(5): 641-650; Mann et al., 1996, in Z. Davidovich & L. A. Norton (Eds.), Biological Mechanisms of Tooth Movement and Craniofacial Adaptation (pp. 7-10). Boston: Harvard Society for the Advancement of Orthodontics).

In some forms, the dental MSCs can be isolated via enzymatic digestion. For example, dental pulp tissue obtained from extracted or naturally exfoliated teeth can be separated from surrounding dentin and subjected to digestion with a collagenase type I and dispase II enzyme solution for approximately 30-60 minutes at 37° C. The digestion releases single cells and small stromal clusters, which can then be filtered through a sterile mesh or cell strainer, centrifuged, and resuspended in a suitable growth medium such as α-minimum essential medium supplemented with fetal bovine serum and antibiotics. The resulting adherent cell population expands in culture and forms a fibroblast-like monolayer characteristic of dental MSCs (e.g., SHED cells).

In some forms, the dental MSCs are isolated via mechanical dissociation of dental pulp tissue. For example, the dental pulp tissue can be aseptically minced into small fragments using sterile scalpels or scissors and directly plated in culture dishes containing nutrient medium. Over several days, cells migrate out from the tissue explants and attach to the culture surface, forming adherent colonies that can be expanded and characterized as dental MSCs. This explant outgrowth method avoids enzymatic digestion and can be useful for clinical-grade cell preparations, as it minimizes exposure to animal-derived enzymes while preserving cell viability and multipotency.

The dental MSCs can be autologous, allogeneic, or xenogeneic, or a combination thereof. In some forms, the dental MSCs are autologous, meaning they are obtained from the same individual to whom the cells are to be administered. For example, pulp tissue or other dental tissue can be collected from a patient's own extracted or exfoliated tooth, and the isolated stem cells can be expanded and, optionally, cryopreserved for subsequent autologous transplantation. Autologous dental MSCs offer the advantage of reduced risk of immune rejection or disease transmission, and are particularly suitable for personalized regenerative therapies, such as dental pulp or periodontal tissue repair in the same patient.

In some forms, the dental MSCs are allogeneic, meaning they are derived from a donor of the same species. For example, stem cells are isolated from the exfoliated or extracted teeth of healthy donors, expanded ex vivo under controlled conditions, and banked for use in multiple recipients. Allogeneic dental MSCs can provide an off-the-shelf source of cells for clinical applications, allowing rapid treatment without requiring autologous cell harvest. In such forms, the allogeneic cells can be matched by blood type or HLA profile or can be used in an immune-privileged context, as dental MSCs possess inherent immunomodulatory properties that reduce the likelihood of graft-versus-host reactions.

In some forms, the dental MSCs are xenogeneic, meaning they are derived from a donor of a different species. For example, dental MSCs can be isolated from porcine, canine, or rodent dental tissues for use in preclinical or translational research models to study stem-cell-mediated tissue regeneration, angiogenesis, or host immune responses. Xenogeneic dental MSCs can be used for proof-of-concept studies, mechanistic investigations, or safety evaluations prior to clinical translation of the corresponding human cell-based therapies.

### 2. Pre-Conditioning Agents

The disclosed dental MSCs (e.g., SHED cells) are pre-treated with one or more pre-conditioning agents capable of inducing a hypoxia-mimicking state under normal oxygen (normoxic) conditions (e.g., about 20-21% O₂, 5% CO₂, and 37° C. in a standard cell culture incubator). Generally, the dental MSCs (e.g., SHED cells) are pre-treated with an effective amount of the pre-conditioning agent for a period of time sufficient to stabilize, activate, or increase expression of HIF-1α relative to a control (e.g., untreated SHED cells cultured under standard normoxic conditions). For example, SHED cells are cultured with an effective amount of the pre-conditioning agent (e.g., TW-37) to trigger downstream cellular pathways involved in angiogenesis, metabolic adaptation, and increased survival in low-oxygen environments.

In some forms, the dental MSCs (e.g., SHED cells) are pre-treated with an effective amount of the pre-conditioning agent to increase angiogenesis relative to a control. For example, the dental MSCs are pre-treated with an effective amount of the pre-conditioning agent (e.g., TW-37) to increase expression and secretion of vascular endothelial growth factor (VEGF). In another example, the dental MSCs are pre-treated with an effective amount of the pre-conditioning agent (e.g., TW-37) to increase expression and secretion of other angiogenic mediators, such as but not limited to angiopoietin-1 (ANGPT1), basic fibroblast growth factor (bFGF or FGF2), platelet-derived growth factor (PDGF-B), hepatocyte growth factor (HGF), and matrix metalloproteinases (e.g., MMP-2 and MMP-9). These factors collectively promote endothelial cell migration, tube formation, and vascular sprouting, resulting in enhanced angiogenic capacity of the pre-conditioned dental MSCs compared to untreated MSCs or other control cells described below.

In some forms, the dental MSCs (e.g., SHED cells) are pre-treated with an effective amount of the pre-conditioning agent to enhance cellular resilience and energy homeostasis relative to a control. For example, the dental MSCs are pre-treated with an effective amount of the pre-conditioning agent (e.g., TW-37) to increase expression of adenylate kinase 4 (AK4), a mitochondrial enzyme involved in maintaining intracellular ATP balance and promoting survival under hypoxic stress. In another example, pre-conditioning of the dental MSCs increases expression of other cytoprotective and metabolic regulators, such as glucose transporter 1 (GLUT1/SLC2A1), pyruvate dehydrogenase kinase 1 (PDK1), and lactate dehydrogenase A (LDHA), which support efficient energy utilization and adaptation to reduced oxygen availability. Collectively, these molecular responses contribute to preservation of ATP homeostasis, reduction of oxidative stress, and increased survival of the pre-conditioned dental MSCs when exposed to ischemic or otherwise hostile environments. As a result, the pre-conditioned cells maintain higher viability and functional activity following transplantation, supporting both tissue regeneration and vascular integration within the dental pulp microenvironment. For example, as demonstrated in the Examples, pre-conditioning dental MSCs (e.g., SHED cells) with the exemplary pre-conditioning agent (e.g., TW-37), triggers stabilization and nuclear accumulation of HIF-1α, even under normal oxygen tension, mimicking the effects of physiological hypoxia. This activation resulted in transcriptional upregulation of VEGF and related angiogenic genes, while increasing expression of AK4, a mitochondrial enzyme that contributes to energy homeostasis and cell survival under hypoxic stress. Unlike conventional HIF-1α stabilizers such as CoCl₂ or DFO, which act as iron chelators and can exhibit cytotoxicity, TW-37 achieved HIF-la stabilization through non-chelating, AK4-mediated mechanisms, thus providing a safer and more controlled pre-conditioning effect.

The control can be an equivalent population of dental MSCs maintained under the same culture conditions but not exposed to the pre-conditioning agent, or alternatively, cells treated with a known HIF-1α stabilizer such as cobalt chloride (CoCl₂) or deferoxamine (DFO) for comparative evaluation of hypoxia-mimetic activity. As demonstrated in the non-limiting Examples, CoCl₂ and DFO are not capable of accurately replicating the hypoxic response in SHED cells, and their effects on HIF-1α stabilization are accompanied by cytotoxicity and inconsistent activation of angiogenic mediators such as VEGF (FIGS. 9A-9F). In comparison, the exemplary pre-conditioning agent, TW-37, induced robust HIF-1α stabilization and AK4 upregulation under normoxic conditions without detectable cytotoxicity, thereby providing a safer and more effective pre-conditioning approach for increasing the angiogenic and survival properties of dental MSCs (FIGS. 1A-1F, 3A-3E, 4A-4E).

i. Exemplary Pre-Conditioning Agents

The preconditioning agent is generally an inhibitor of the B-cell lymphoma-2 (Bcl-2) family (referred to as “Bcl-2 inhibitor”). The Bcl-2 protein family is a group of intracellular regulatory proteins that control the mitochondrial apoptotic pathway and cell survival under stress. Members of this family include both anti-apoptotic proteins (e.g., Bcl-2, Bcl-XL, Mcl-1, and Bcl-w) and pro-apoptotic proteins (e.g., Bax, Bak, Bad, and Bid). Inhibitors of the anti-apoptotic members function by disrupting the interaction between pro- and anti-apoptotic Bcl-2 proteins. More detailed descriptions of Bcl-2 inhibitors, including the description of exemplary Bcl-2 inhibitors and their mechanism of actions can be found in Xu, et al. (2023), Cancers, 15:4957; Zhang, et al. (2022), European Journal of Medicinal Chemistry, 232:114184; U.S. Publication No. 2010/0305122 to Bruncko et al.; U.S. Publication No. 2010/0152183 to Bruncko et al.; and U.S. Publication No. 2007/0027135 to Bruncko et al. the contents of which are incorporated herein by reference in their entireties.

It has been found that treatment of dental MSCs (e.g., SHED cells) with an exemplary Bcl-2 family inhibitor can activate adaptive cellular responses associated with enhanced survival and angiogenic potential. As demonstrated in the Examples, such pretreatment promotes stabilization of HIF-1α under normoxic conditions and upregulation of adenylate kinase 4 (AK4), a mitochondrial enzyme that contributes to maintenance of ATP homeostasis and cell viability during stress (FIGS. 1A-1F, 3A-3E, 4A-4E).

The exemplary compound TW-37, a small-molecule inhibitor originally designed to target the Bcl-2 protein family, was unexpectedly found to act as an effective pre-conditioning agent for dental MSCs (FIGS. 1A-1F, 3A-3E, 4A-4E). In SHED cells, TW-37 induced HIF-1α stabilization and increased AK4 expression in a dose- and time-dependent manner, leading to marked increases in VEGF secretion and enhancement of angiogenic functions, including endothelial sprouting and tube formation in vitro (FIGS. 4A-4E). Importantly, these effects were achieved under normoxic culture conditions and were not replicated by conventional hypoxia-mimetic compounds such as cobalt chloride (CoCl₂) or deferoxamine (DFO) (FIGS. 9A-9F).

Therefore, any Bcl-2 inhibitor that can induce or enhance stabilization of HIF-1α, either directly or indirectly (e.g., through modulation of AK4 expression or other hypoxia-responsive pathways), is suitable for use as the pre-conditioning agent, provided that it promotes angiogenic and cytoprotective effects in dental MSCs without causing significant cytotoxicity under normoxic culture conditions.

Suitable small-molecule Bcl-2 inhibitors that can be used as the pre-conditioning agent include, but are not limited to, TW-37, ABT-737, ABT-263 (Navitoclax), BM-1197, S44563, AZD4320, ABT-199 (Venetoclax), S55746, APG-2575 (lisaftoclax), G3139 (oblimersen), AZD0466, APG-1252 (pelcitoclax), S44563, GX15-070 (obatoclax), AT-101, or pharmaceutically acceptable salts, solvates, or derivatives thereof. All of these compounds are currently either in Phase 1/2/3 clinical trials or FDA-approved. These compounds, or combinations thereof, can be used to achieve similar stabilization of HIF-1α and induction of angiogenic signaling in dental MSCs. In preferred forms, the Bcl-2 inhibitor is TW-37 ([(−)-gossypol acetic acid derivative]; chemically designated as 2-amino-9-(10H-phenothiazin-10-yl)-9H-thioxanthen-9-ol). The structures of exemplary small-molecule Bcl-2 inhibitors that can be used as the pre-conditioning agent are provided in Table 1 below.

TABLE 1
Exemplary Pre-Conditioning Agents

| Bcl-2 | |
|---|---|
| Inhibitor | Chemical Structure |
| TW-37 | 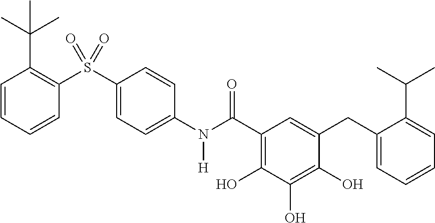 [SMILES: CC(C)c1ccccc1Cc1cc(C(=O)Nc2ccc(S(=O)(=O)c3ccccc3C(C)(C)C)cc2)c(O)c(O)c1O] |
| ABT-737 | 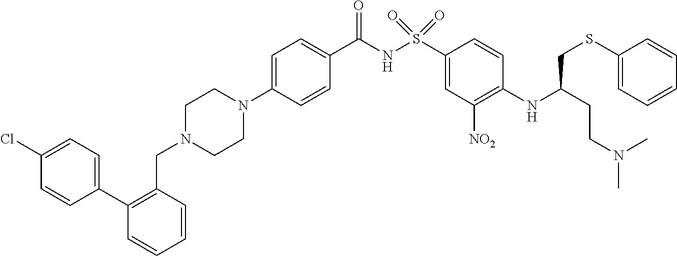 [SMILES: CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]] |
| ABT-263 (Navitoclax) | 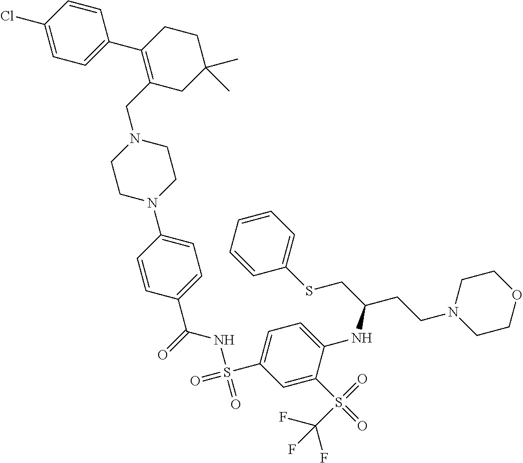 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| BM-1197 | 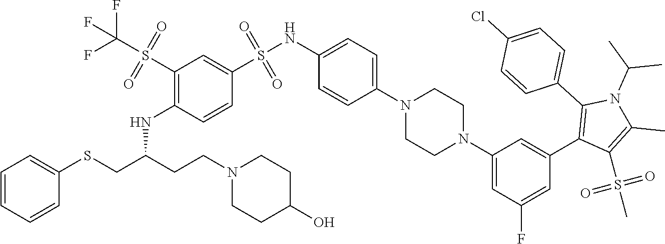 [SMILES: Cc1c(S(C)(=O)=O)c(-c2cc(F)cc(N3CCN(c4ccc(NS(=O)(=O)c5ccc(N[C@H](CCN6CCC(O)CC6)CSc6ccccc6)c(S(=O)(=O)C(F)(F)F)c5)cc4)CC3)c2)c(-c2ccc(Cl)cc2)n1C(C)C] |
| S44563 | 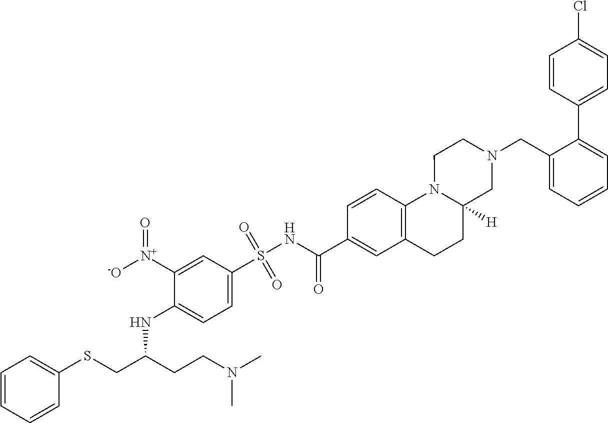 |
| AZD4320 | 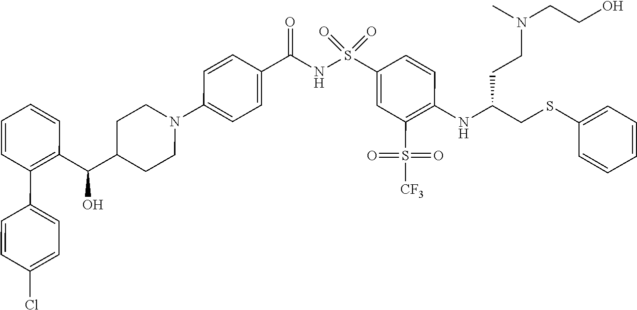 [SMILES: CN(CCO)CC[C@H](CSc1ccccc1)Nc1ccc(S(=O)(=O)NC(=O)c2ccc(N3CCC([C@@H](O)c4ccccc4-c4ccc(Cl)cc4)CC3)cc2)cc1S(=O)(=O)C(F)(F)F] |
| ABT-199 (Venetoclax) | 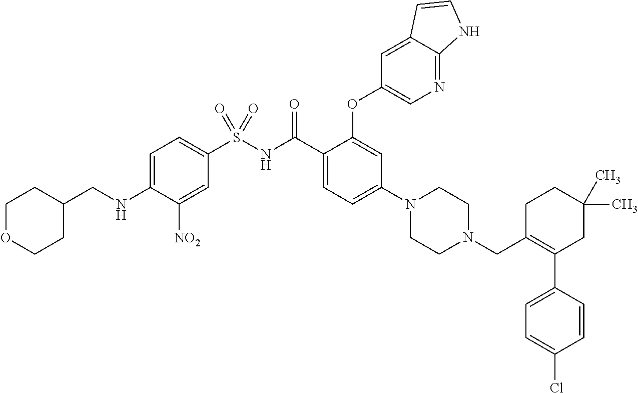 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1] |
| S55746 (BLC201) | 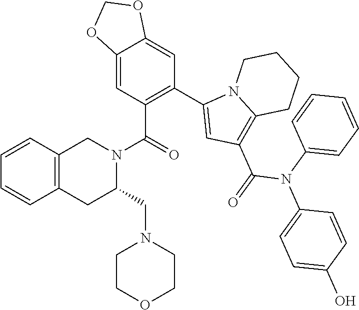 [SMILES: O=C(c1cc(-c2cc3c(cc2C(=O)N2Cc4ccccc4C[C@H]2CN2CCOCC2)OCO3)n2c1CCCC2)N(c1ccccc1)c1ccc(O)cc1] |
| APG-2575 (lisaftoclax) | 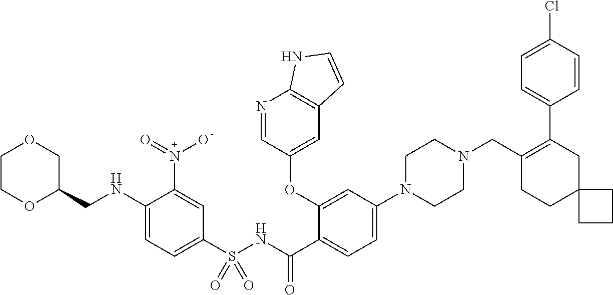 |
| APG-1252 (pelcitoclax) | 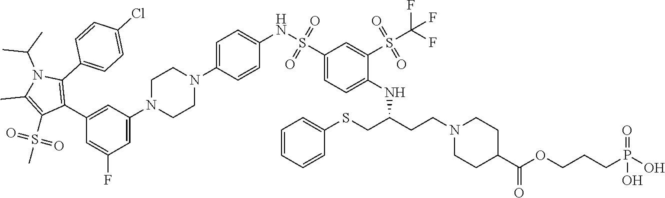 [SMILES: Cc1c(S(C)(=O)=O)c(-c2cc(F)cc(N3CCN(c4ccc(NS(=O)(=O)c5ccc(N[C@H](CCN6CCC(C(=O)OCCCP(=O)(O)O)CC6)CSc6ccccc6)c(S(=O)(=O)C(F)(F)F)c5)cc4)CC3)c2)c(-c2ccc(Cl)cc2)n1C(C)C] |
| S44563 | 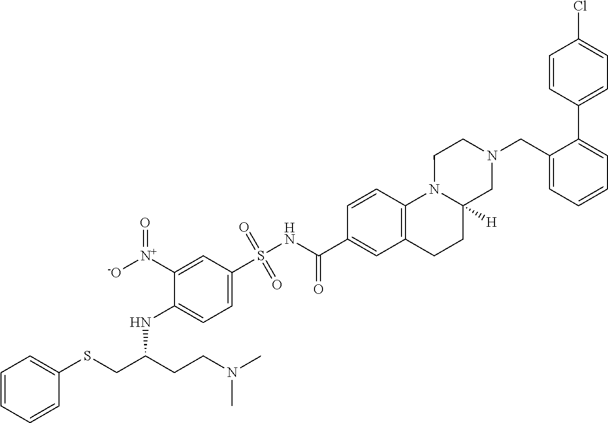 |
| GX15-070 (obatoclax) | 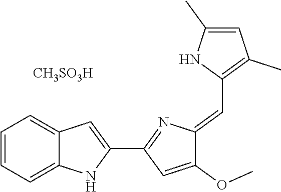 [SMILES: COC1=CC(c2cc3ccccc3[nH]2)=N/C1=C\c1[nH]c(C)cc1C ; "CH3SO3H"] |
| AT-101 | 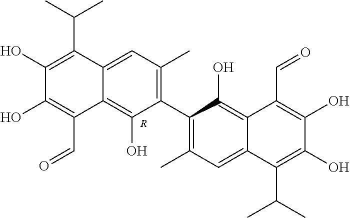 |

In some forms, the pre-conditioning composition includes an effective amount of a single Bcl-2 inhibitor capable of stabilizing HIF-1α and enhancing the angiogenic and survival properties of dental MSCs. For example, the dental MSCs can be pre-treated with an effective amount of TW-37, such as between about 1 μM and 20 μM, for a defined duration (e.g., 3 to 24 hours) under normoxic conditions to induce AK4 upregulation, VEGF secretion, and improved tolerance to hypoxic stress.

In some forms, two or more (e.g., three, four, or five) Bcl-2 inhibitors are used together as pre-conditioning agents to broaden or strengthen the cellular response associated with HIF-1α stabilization and angiogenic activation. For example, TW-37 is used in combination with another small-molecule Bcl-2 family inhibitor, such as ABT-737, ABT-263 (Navitoclax), ABT-199 (Venetoclax), BM-1197, S44563, AZD4320, or S55746, or a pharmaceutically acceptable salt, solvate, or derivative thereof. In such forms, the Bcl-2 inhibitors can be administered concurrently or sequentially to the dental MSCs during the pre-conditioning phase, and the respective concentrations and exposure durations can be selected to achieve enhanced HIF-1α stabilization, increased AK4 expression, and augmented VEGF secretion, while maintaining high cell viability under normoxic culture conditions.

ii. Exposure Parameters

The dosage and exposure parameters can be changed according to cell type, culture density, and medium composition to achieve maximal HIF-1α stabilization while maintaining healthy proliferative potential.

The dental MSCs can be cultured with an effective amount of the pre-conditioning agent to induce hypoxia-like responses without compromising cell viability. In some forms, the effective amount of the pre-conditioning agent ranges from about 0.1 μM to about 50 μM. For example, the effective amount of pre-conditioning agent ranges from about 1 μM to about 45 μM, from about 1 μM to about 40 μM, from about 1 μM to about 40 μM, from about 1 μM to about 35 μM, from about 1 μM to about 30 μM, from about 1 μM to about 25 μM, from about 1 μM to about 20 μM, from about 1 μM to about 15 μM, from about 1 μM to about 10 μM, from about 1 μM to about 5 μM, from about 5 μM to about 40 μM, from about 5 μM to about 30 μM, from about 5 μM to about 25 μM, to about 5 μM to about 20 μM, from about 5 μM to about 10 μM, from about 10 μM to about 40 μM, from about 10 μM to about 30 μM, or from about 10 μM to about 20 μM. For example, the effective amount of the pre-conditioning agent is about 1 μM, about 2μ, about 5μ, about 7μ, about 10μ, about 15μ, about 20μ, about 25μ M, about 30 μM, about 35 μM, about 40 μM, or about 50 μM.

The dental MSCs (e.g., SHED cells) are cultured in an effective amount of the pre-conditioning agent (e.g., TW-37) for a duration of time effective to induce hypoxia-like responses without compromising cell viability. In some forms, the duration of time effective to induce hypoxia-like responses in the dental MSCs range from about 2 hours to about 48 hours. For example, the duration of time effective to induce hypoxia-like responses in the dental MSCs range from about 2 hours to about 40 hours, from about 2 hours to about 36 hours, from about 2 hours to about 30 hours, from about 2 hours to about 24 hours, from about 2 hours to about 18 hours, from about 2 hours to about 12 hours, from about 6 hours to about 48 hours, from about 6 hours to about 40 hours, from about 6 hours to about 34 hours, from about 6 hours to about 24 hours, from about 6 hours to about 18 hours, from about 6 hours to about 12 hours, such as between about 3 hours and about 24 hours. In one example, the duration of time effective to induce hypoxia like responses in the dental MSCs range from about 6 hours to about 12 hours.

Optionally, the dental MSCs are pre-treated with one or more additional active agents in conjunction with the pre-conditioning agent to enhance, complement, or support the biological effects achieved during pre-conditioning. Such additional active agents can be selected to promote cell survival, angiogenesis, metabolic resilience, or antioxidative capacity of the dental MSCs prior to transplantation.

Optionally, one or more additional active agents are included with the pre-conditioning agent to potentiate the induction of hypoxia-responsive and cytoprotective pathways. For example, TW-37 can be applied to the dental MSCs together with another HIF-1α stabilizer (e.g., cobalt chloride (CoCl₂), deferoxamine (DFO), or a prolyl hydroxylase domain (PHD) inhibitor) to amplify HIF-1α activation and downstream signaling of angiogenic mediators such as VEGF. In other forms, the pre-conditioning agent is combined with a cell-protective modulator, such as N-acetylcysteine, resveratrol, melatonin, erythropoietin, or basic fibroblast growth factor (bFGF), to enhance antioxidant defenses, reduce oxidative stress, or promote endothelial cell recruitment.

When used, the additional active agents can be applied concurrently or sequentially during the pre-conditioning phase, either in the same culture medium or in separate incubation steps, depending on the desired biological response. One of skill in the art can titrate and select the appropriate concentrations and exposure durations of the one or more additional active agents to achieve activation of angiogenic, survival, and regenerative pathways, while maintaining high viability and functional potency of the dental MSCs.

### 3. Pharmaceutical Carriers and Excipients

The disclosed compositions containing the pre-conditioned dental MSCs can be formulated with one or more pharmaceutically acceptable carriers, excipients, or delivery media suitable for maintaining cell viability, stability, and functionality prior to administration. Once formulated with a pharmaceutically acceptable carrier, the dental MSCs are referred to as the “pharmaceutical formulation”.

As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active agents (e.g., compounds such as CoCl₂ and/or DFO) can also be incorporated into compositions. For example, in the context of the dental MSCs, a pharmaceutically acceptable carrier” can be any physiologically compatible vehicle, solution, or matrix that does not adversely affect the biological properties of the stem cells or the efficacy of the pre-conditioning agent.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The pharmaceutically acceptable carrier can include an aqueous medium suitable for maintaining viable cells, such as phosphate-buffered saline (PBS), Hank's balanced salt solution (HBSS), Ringer's solution, culture medium (e.g., α-MEM or DMEM), or a cryopreservation solution containing agents such as but not limited to dimethyl sulfoxide (DMSO), human serum albumin, or trehalose. Formulations of the dental MSCs can further include stabilizers (e.g., human serum albumin, gelatin, or trehalose), osmoprotectants (e.g., mannitol, sucrose, or glycerol), buffering agents (e.g., phosphate-buffered saline (PBS), HEPES, or Tris buffer), antioxidants (e.g., ascorbic acid, glutathione, or N-acetylcysteine), or antimicrobial agents (e.g., gentamicin, amphotericin B, or penicillin-streptomycin), to preserve the structural and functional integrity of the cells during storage, transport, and handling.

In some forms, the pre-conditioned dental MSCs are formulated in a liquid suspension, gel, or semi-solid matrix suitable for topical, injectable, or implantable delivery to a target tissue site, such as the dental pulp cavity, alveolar bone, periodontal ligament, or soft tissue defect. Exemplary matrices include collagen gels, fibrin gels, alginate hydrogels, hyaluronic acid matrices, or other biocompatible scaffolds that support cell adhesion and vascular integration following administration.

In other forms, the pre-conditioning agent(s) and the dental MSCs are provided separately, allowing the pre-conditioning step to be performed at the point of care prior to administration. For example, the composition can be supplied as a kit containing (i) a vial of viable or cryopreserved dental MSCs, and (ii) a separate vial of the pre-conditioning agent (e.g., a Bcl-2 inhibitor such as TW-37) in a pharmaceutically acceptable solution. Upon receipt, the cells can be thawed or reconstituted and then exposed ex vivo to the pre-conditioning agent under controlled conditions (e.g., defined concentration and incubation period) to induce HIF-1α stabilization and angiogenic activation prior to transplantation.

Optionally, the pharmaceutical formulation includes additives that support post-transplantation survival and vascularization, such as growth factors (e.g., VEGF, FGF2, PDGF-B), cytokines (e.g., IL-6, IL-8), or extracellular matrix proteins (e.g., laminin, fibronectin). The formulations can be sterilized, packaged in single-use containers, and stored under refrigerated or cryogenic conditions according to standard cell therapy manufacturing practices.

### 4. Dental Formulations

The pharmaceutical formulations of the pre-conditioned dental MSCs can be formulated in a variety of dental-specific and clinically applicable delivery formats suitable for storage, handling, and administration to a target tissue site (e.g., dental pulp cavity, dentin-pulp interface, and root canal space). Suitable dental formulations include cell suspensions, cell-seeded scaffolds or gels, lyophilized or cryopreserved cell formulations, and encapsulated or microcarrier-based formulations.

In some forms, the pre-conditioned dental MSCs are provided as a cell suspension in a physiologically compatible carrier (e.g., PBS, Ringer's solution, or a basal culture medium, such as α-MEM or DMEM)) supplemented with serum or serum-free stabilizers. The cell suspension can include cryoprotectants (e.g., DMSO, trehalose, or human serum albumin) and/or antioxidants to maintain viability during storage and transport. For example, the cells can be freshly pre-conditioned prior to shipment or cryopreserved in single-use vials for thawing and immediate clinical application. Such suspensions can be administered directly to dental pulp cavities, root canals, periodontal pockets, bone defects, or gingival soft tissues via appropriate delivery methods such as syringe injection or micro-catheter delivery.

In other forms, the pre-conditioned dental MSCs are incorporated into a biocompatible scaffold or hydrogel matrix that provides a three-dimensional environment supporting cell adhesion, proliferation, and vascularization following implantation. Suitable matrices include collagen, fibrin, alginate, hyaluronic acid, chitosan, gelatin, Matrigel®, polylactic acid (PLA), polyglycolic acid (PGA), and their copolymers (PLGA), among others. The scaffold can be pre-loaded with growth factors, such as vascular endothelial growth factor (VEGF), basic fibroblast growth factor (bFGF), or platelet-derived growth factor-B (PDGF-B), to sustain angiogenic signaling and promote tissue integration in vivo. In some forms, the matrix is injectable and self-crosslinking, allowing minimally invasive administration into confined spaces such as the root canal or periapical lesion. In other forms, the matrix can be provided as a solid or semi-solid plug, sponge, or membrane configured to fill bone or soft-tissue voids, conform to the defect shape, and gradually degrade as new, vascularized tissue forms.

For extended shelf life and distribution, the pre-conditioned dental MSCs can be cryopreserved or lyophilized in the presence of suitable cryoprotective agents. In some forms, the cells are cryopreserved and stored in liquid nitrogen or ultra-low temperature freezers, then reconstituted immediately prior to use to permit high post-thaw viability. In other forms, the pre-conditioned dental MSCs are lyophilized, meaning they are dried under controlled vacuum conditions in the presence of protective sugars such as trehalose or sucrose, and subsequently rehydrated with sterile water or buffer before clinical use. These formulations provide long-term stability, sterility, and transportability, while preserving the ability of the MSCs to adhere, proliferate, and differentiate following reconstitution, thereby maintaining their functional and therapeutic potential.

In some forms, the pre-conditioned dental MSCs are encapsulated within microcapsules, microspheres, or nanoparticles composed of biocompatible polymers such as alginate, PLGA, gelatin, or polyethylene glycol (PEG). This encapsulation provides mechanical protection to the cells during delivery and allow the controlled release of paracrine factors at the target site, thereby enhancing localized angiogenic and regenerative effects. Alternatively, the pre-conditioned dental MSCs can be expanded on microcarriers in suspension culture and subsequently delivered as cell-laden microbeads, which are particularly suitable for placement into root canal systems, alveolar bone voids, or periodontal defects. These microcarrier-based formats support uniform cell distribution and integration within the host tissue.

For regenerative endodontic and periodontal applications, the formulations can also be adapted for direct topical or injectable delivery. In some forms, topical gels or pastes containing pre-conditioned MSCs can be applied into root canals, extraction sockets, or gingival recessions to promote tissue regeneration and accelerate healing. In other forms, injectable suspensions or hydrogels containing the pre-conditioned dental MSCs can be delivered using standard dental syringes or micro-needles, providing a minimally invasive approach to regenerate dental pulp, alveolar bone, or soft tissue. In some forms, the formulations further include radiopaque agents (e.g., barium sulfate) or color indicators to assist in guided placement and clinical visualization during application.

In some forms, the dental MSCs and the pre-conditioning agent (e.g., TW-37 or another Bcl-2 inhibitor) are supplied as separate components of a kit. The kit can include: (i) a vial of viable or cryopreserved dental MSCs, and (ii) a vial of the pre-conditioning agent formulated in a sterile solution. Upon receipt, the clinician or technician may reconstitute or thaw the cells, then expose them to the pre-conditioning agent for a defined period under normoxic conditions before administering the activated cells to the patient's target tissue site. This two-component configuration allows on-demand pre-conditioning, ensuring that the MSCs exhibit maximal biological potency and viability immediately prior to transplantation.

### C. Methods of Pretreating and/or Preconditioning Dental MSCs

Methods of pre-treating or pre-conditioning dental MSCs (e.g., SHED cells) are provided. The methods are suited to increasing the survival, angiogenic potential, and regenerative capacity of the dental MSCs (e.g., SHED cells) prior to transplantation. These methods generally involve ex vivo exposure of the dental MSCs to one or more pre-conditioning agents (e.g., small-molecule Bcl-2 inhibitors such as TW-37) under controlled normoxic conditions, for a defined duration and concentration sufficient to stabilize hypoxia-inducible factor-1 alpha (HIF-1α), upregulate angiogenic mediators (e.g., VEGF), and increase resistance to hypoxic or oxidative stress, compared to a control, following implantation in a subject.

### 1. Methods of Pretreating/Precondition In Vitro

In some forms, the method for enhancing cell survival and promoting neovascularization and/or vascularized tissue regeneration following transplantation requires administering an effective amount of a compound that stabilizes or activates or expresses hypoxia-inducible factor-1 alpha (HIF-1α). In some forms, the compound is an inhibitor of Bcl-2 family inhibitor. In some forms, the inhibitor of Bcl-2 family is a small molecule, TW-37.

An exemplary method for pre-treating dental MSCs (e.g., SHED cells) includes: (i) administering in vitro an effective amount of a pre-conditioning agent (e.g., a Bcl-2 inhibitor such as TW-37) to the dental MSCs under normoxic culture conditions for a defined period sufficient to stabilize HIF-1α and enhance secretion of angiogenic factors (e.g., VEGF, ANGPT1, or FGF2). The method can further include (ii) washing and/or resuspending the pre-conditioned dental MSC cells in a sterile carrier medium (e.g., phosphate-buffered saline, culture medium, or a cryoprotective solution).

i. HIF-1α

HIF-1α is the active subunit of the HIF family, which is a heterodimeric complex containing α & beta (β) subunits (HIF-1α, HIF-2α, or HIF-3α and HIF-β)^{[8]}. In normoxic conditions, the a subunits of HIF can be hydroxylated at conserved proline residues by HIF prolyl-hydroxylases (PHD) or be inactivated by the factor-inhibiting HIF-1α (FIH) in the way of hydroxylation at asparagine residue, which eventually leads to HIF-1α's degradation^{[8,9]}. However, in hypoxia, HIF-1α is heavily activated, and the degradation is inhibited since hypoxia significantly suppresses the activity of PHD or FIH, which results in the accumulation of HIF-1α in the nucleus^{[8,9]}. Sufficient studies have provided convincing evidence of a strong correlation between HIF-1α stabilization and angiogenesis, metabolism, erythropoiesis, cell survival, cell proliferation, tumor resistance therapy, organ protection, and so on^{[5-7,10]}.

Generally, there are three methods of HIF-1α stabilization: PHD knockdown, custom peptides to block HIF-1α degradation, and the use of chemical compounds to target PHD or FIH, in which chemical method has been increasingly explored as a safer and clinically applicable strategy for stabilizing HIF-1α in cell-based therapies as the lentiviral-mediated gene knockdown methods that was previously used cannot be applied in the clinical setting due to safety concerns. So it is important to explore optimal and safe methods that can be used clinically. The chemical compounds, like cobalt chloride (CoCl₂) and desferrioxamine (DFO), currently being examined to activate HIF-1α expression in cells, are associated with low efficacy and high cytotoxicity^{[16-19]}. Furthermore, it has been shown that each of these compounds also has distinct mechanisms of action, which can also influence stem/progenitor cell properties and fate differentially^{[16,18,19]}. Therefore, it is crucial to identify and characterize the optimal compound(s) and dosage setting for applying the hypoxic preconditioning phenomenon in SHED.

ii. TW-37

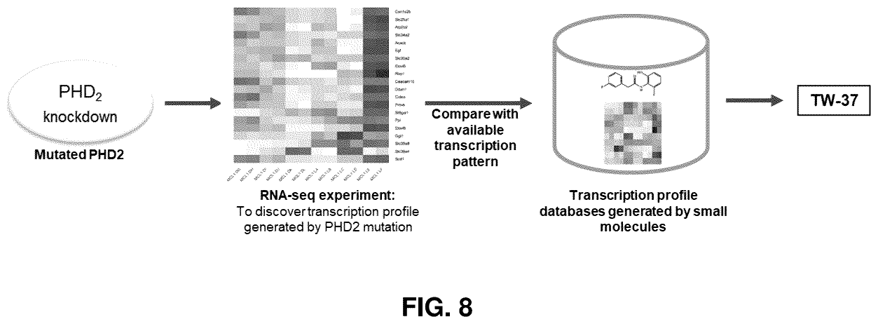

In search of optimal small molecules that can be used on SHED, RNA sequencing was performed for PHD₂ knockdown-SHED, and the data were processed to extract upregulated/downregulated genes in a previous study^{[14]}. Using a bioinformatics approach, the transcript profile was compared with the unique signature profile of a drug to create a connectivity map (cMAP) database. Using this approach, based on the connectivity score and p-value that can potentially mimic the transcriptomic signature, it was identified that the small molecule TW-37 can have an influence on HIF-1α expression in SHED (FIG. 8).

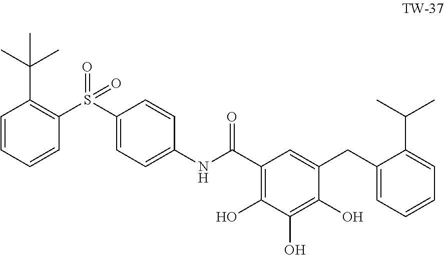 [SMILES: CC(C)c1ccccc1Cc1cc(C(=O)Nc2ccc(S(=O)(=O)c3ccccc3C(C)(C)C)cc2)c(O)c(O)c1O]

TW-37 is a small molecule inhibitor of Bcl-2 designed using a structure-based design strategy with an anti-cancer role and functions as a potential apoptosis-inducing agent for the treatment of cancers^{[20-22]}. TW-37 functions as a HIF-1α stabilizer through regulating adenylate kinase 4 (AK4) in SHED. TW-37 is a potent small-molecule inhibitor that attenuates Bcl-2 activation and inhibits multiple Bcl-2 family members including Bcl-xL and Mcl-1. TW-37 also inhibits in vivo and in vitro the activation of Jagged-1 and Notch-1.

Previous research on TW-37 focuses on its anti-cancer roles and functions as a potential apoptosis-inducing agent for the treatment of cancers. However, the present invention discloses a previously unidentified function of TW-37 as a HIF-1α stabilizer in SHED. HIF-1α stabilization under normoxia achieved by TW-37 is a valuable approach to enhance the therapeutic potential of dental stem cells through augmentation of the angiogenic properties and post-implantation cell survival.

HIF-1α stabilizers, also known as hypoxia-mimetic agents, refer to the pharmacological or chemical agents or compounds that promote HIF-1α expression and stimulate the situation of hypoxia preconditioning. Cobalt chloride (CoCl₂) and desferrioxamine (DFO) are the most studied and used HIF-1α stabilizers in dental stem cells^{[23-25]}. In the current study, CoCl₂ and DFO were examined to activate HIF-1α expression in SHED with low efficacy and high cytotoxicity (FIGS. 9A-9F).

Besides, most of the previous HIF-1α stabilizers in the literature function as PHD inhibitors. However, in the instant application, the difference is that TW-37 stabilizes HIF-1α expression by regulating AK4, which is a key modulator enzyme of ATP and maintains the homeostasis of cellular nucleotides.

TW-37 can be purchased commercially, and the CAS No. is 877877-35-5. It can be dissolved in dimethylsulfoxide and used in different concentrations according to different cell types and experimental models. In this invention, at first, six concentrations (0 μM, 1 μM, 2.5 μM, 5 μM, 10 μM, 20 μM) were when applied to SHED and was found that TW-37 had a concentration-dependent effect on HIF-1α stabilization. Based on the results, a 10 μM concentration of TW-37 was applied in the following study of RNA sequencing and regulation mechanism. The small molecule can be added into the cell culture medium directly or used within a drug delivery system such as a hydrogel which may be better for the in vivo exploration.

Any of the additional Bcl-2 inhibitors described above (e.g., ABT-263 (Navitoclax), ABT-199 (Venetoclax)), can be used as an alternative or in conjunction with TW-37.

iii. Effective Amounts

In some forms, the method for enhancing cell survival and promoting neovascularization and/or vascularized tissue regeneration following transplantation requires pretreating cells prior to transplantation with an optimal dosage of the compound, thereby inducing hypoxic preconditioning under normoxic conditions. In some forms, the pretreatment of the cells with the compound facilitates the expression, stabilization, or activation of HIF-1α during normoxia, resulting in the angiogenic/vasculogenic response. In some forms, the compound can be used within a drug delivery system such as but not limited to a hydrogel for stabilizing or activating HIF-1α in vivo.

In some forms, the concentration of compound used for preconditioning or pretreating can range from about 0.1 μM-50 μM, 0.3 μM-45 μM, 0.6 μM-40 μM, 0.9 μM-35 μM, 1.5 μM-30 μM, 3 μM-25 μM, 5 μM-20 μM, 10 μM-15 μM, preferably 1 μM-20 μM.

The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The efficacy of administration of a particular dose of the compounds or compositions according to the methods described herein can be determined by evaluating the particular aspects of the medical history, signs, symptoms, and objective laboratory tests that are known to be useful in evaluating the status of a subject in need for transplantation. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved, (2) the progression of the disease or condition is shown to be stabilized, or slowed, or reversed, or (3) the need for other medications for treating the disease or condition is lessened or obviated, then a particular treatment regimen will be considered efficacious.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

Any of the compounds having the formula of the compound can be used therapeutically in combination with a pharmaceutically acceptable carrier. The compounds described herein can be conveniently formulated into pharmaceutical compositions composed of one or more of the compounds in association with a pharmaceutically acceptable carrier. See, e.g., Remington's Pharmaceutical Sciences, latest edition, by E. W. Martin Mack Pub. Co., Easton, PA, which discloses typical carriers and conventional methods of preparing pharmaceutical compositions that can be used in conjunction with the preparation of formulations of the compounds described herein. These most typically would be standard carriers for administration of compositions to humans. In one aspect, humans and non-humans, including solutions such as sterile water, saline, and buffered solutions at physiological pH. Other compounds will be administered according to standard procedures used by those skilled in the art.

The pharmaceutical compositions described herein can include, but are not limited to, carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The compounds and pharmaceutical compositions described herein can be administered to the subject in a number of ways depending on whether local or systemic treatment is desired, and on the area to be treated. Moreover, a pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. Parenteral administration, if used, is generally characterized by injection and includes intravenous (IV), subcutaneous (SC), intramuscular (IM), epidural and intra-articular injection, as well as surgical insertion of depots in the organ or tissue of interest (Bittner, et al., BioDrugs, 32:425-440 (2018); Lee et al., J. Pharm. Investig., 49:459-476 (2019); Chaudhary et al., Crit. Rev. Ther. Drug Carrier Syst., 36:137-181 (2019)). Injectables can be prepared in conventional forms, either as liquid solutions or suspensions, solid forms suitable for solution or suspension in liquid prior to injection, or as emulsions (Park et al., J Control Release, 342:53-65 (2022); Nkanga, et al., Advanced Drug Delivery Reviews, 167:19-46, (2020); Sheikh, et al., Asian Journal of Pharmaceutics, 10(4):S465-S471 (2016); Rhee et al., Pharmaceutical Technology Drug Delivery, p. S6 (2010)). An exemplary approach for parenteral administration involves use of a slow release or sustained release system such that a constant dosage is maintained. See for example, U.S. Pat. No. 9,700,630, WO 2006/125620, and KR 101898816.

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration can include powders or granules, suspensions or solutions in water or non-aqueous media, capsules, sachets, or tablets. Thickeners, flavorings, diluents, emulsifiers, dispersing aids or binders can be desirable.

### 2. Cells to be Pretreated

In some forms, the cells pretreated for transplantation include stem cells. In some forms, the stem cells can be obtained from human exfoliated deciduous teeth (SHED). In some forms the stem cells can be mesenchymal stem cells (MSCs), hematopoietic stem cells (HSCs), neural stem cells (NSCs), cardiac stem cells (CSCs), induced pluripotent stem cells (iPSCs), endothelial progenitor cells (EPCs) or adipose-derived stem cells (ADSCs).

Stem cells from human exfoliated deciduous teeth (SHED) have been identified as a population of highly proliferative and clonogenic dental stem cells that are able to induce bone formation, dentin regeneration, neuroregeneration, and angiogenesis^{[11]}. Due to the superior stem cell properties and the non-invasive nature of the source from exfoliated teeth, SHED have become important cells in stem cell-based autologous transplantation and tissue engineering^{[11,12]}. More interestingly, it has been shown that HIF-1α expression in SHED is significantly stabilized in normoxia following the knockdown of prolyl hydroxylase domain, which in return improves the angio-/vasculogenic properties and post-implantation cell survival^{[13-15]}.

In some forms, the pretreated cells can be transplanted into a subject using various methods, depending on the treatment goals and target tissues. Some of the methods include but are not limited to intravenous (IV) injection, intramuscular (IM) injection, intra-articular injection, intrathecal injection, direct tissue injection, intra-arterial injection, subcutaneous injection, topical application, or scaffold-based transplantation.

### D. Methods of Using the Pre-Conditioned Dental MSCs

Methods for enhancing cell survival and promoting neovascularization and/or vascularized tissue regeneration following transplantation in a subject are disclosed.

The methods include the use of disclosed pre-conditioned dental MSCs (e.g., SHED cells) and/or formulations thereof, for increasing tissue regeneration, angiogenesis, and repair of dental and craniofacial defects. The methods include administering an effective amount of the pre-conditioned dental MSCs to a subject in need thereof, to restore, repair, or regenerate damaged or diseased dental tissues.

The subject in need can be a subject having a dental injury such as a dental pulp injury, a periapical lesion, periodontal defect, alveolar bone loss, or maxillofacial soft-tissue defect.

The pre-conditioned dental MSCs can be administered alone, in combination with a carrier or scaffold, or as part of a formulated composition (e.g., injectable suspension, hydrogel, or matrix-embedded construct).

### 1. Dental Diseases/Conditions to be Treated

In preferred forms, the compounds that stabilizes HIF-1α under normoxia can be used to enhance the therapeutic potential of dental stem cells for dental pulp regeneration through augmentation of the angiogenic properties and post-transplantation cell survival. Dental pulp or tooth pulp is the innermost layer of a tooth. It contains nerves, blood vessels, specialized cells and connective tissues, which provide nutrients for the tooth. Dental pulp is what keeps the tooth alive. The outer layers of the tooth, dentin and enamel, protect the pulp of the tooth. But the dental pulp can become exposed due to cavities, cracks or chronic teeth grinding (bruxism). The dental pulp performs several functions that contribute to the health and maintenance of the tooth such as formation of dentin, nourishment, sensation, defense and repair, and immune response.

In some forms, the compounds can be used to treat conditions that typically involve reduced blood supply, where new blood vessel formation (neovascularization) could help restore oxygen and nutrient delivery to affected tissues such as, but not limited to, periodontal disease, tooth pulp necrosis, alveolar bone loss, cleft palate and cleft lip, dental implant integration (osseointegration), oral mucosal defects, jawbone defects, peripheral artery disease (PAD), coronary artery disease (CAD), stroke, ischemic heart disease (IHD), diabetic foot ulcers, chronic wounds, critical limb ischemia, retinopathy of prematurity (ROP), ischemic stroke, myocardial infarction, ischemic cardiomyopathy, ischemic colitis, wound healing disorders, vascular dementia, or Buerger's disease (thromboangiitis obliterans).

### 2. Methods of Treatment

Administration of the pre-conditioned cells can be performed via direct injection, surgical placement, or topical application, depending on the target site and clinical indication.

In some forms, injectable formulations of pre-conditioned dental MSCs are administered using standard dental syringes or micro-needles into soft or hard tissue defects, allowing minimally invasive delivery. For example, in regenerative endodontic therapy, the injectable suspension can be introduced into the apical region of an immature tooth to promote apex closure and revascularization. In other cases, the pre-conditioned cells are applied as a topical gel or film over exposed root surfaces, extraction sockets, or gingival wounds to enhance soft-tissue healing.

In some forms, the pre-conditioned dental MSCs are administered together with a scaffold or hydrogel pre-loaded with growth factors, such as VEGF, bFGF, PDGF, or HGF, to sustain pro-angiogenic and pro-survival signaling after transplantation.

The dosage of pre-conditioned cells per application depends on a variety of factors, including but not limited to the size and severity of the defect. In some forms, the dosage of the pre-conditioned cells that is administered to the subject can range from about 1×10² to 1×10¹⁰ viable cells per application. For example, the dosage of the pre-conditioned dental MSCs that is administered to the subject is about 1×10² viable cells, about 1×10³ viable cells, about 1×10⁴ viable cells, about 1×10⁵ viable cells, about 1×10⁶ viable cells, about 1×10⁷ viable cells, about 1×10⁸ viable cells, or about 1×10⁹ viable cells per application. The administration can be performed as a single treatment or in multiple sessions, depending on clinical progress and regenerative needs.

The pre-conditioned dental MSCs (e.g., SHED cells), can be administered to a subject in need of dental pulp regeneration, periodontal regeneration, and/or alveolar bone or maxillofacial bone regeneration.

In one example, when administered to a subject in need for dental pulp regeneration, the pre-conditioned SHED cells can be delivered directly into the root canal space of a disinfected tooth. For example, the tooth can be isolated and the root canal is irrigated to remove necrotic tissue. The root canal is then dried, and the pre-conditioned dental MSC suspension is injected or placed into the pulp chamber, optionally together with a biocompatible scaffold (e.g., collagen, fibrin, or hyaluronic acid hydrogel) that supports cell attachment and migration. The root canal is subsequently sealed with a restorative material such as mineral trioxide aggregate (MTA) or a resin composite. The implanted SHED cells differentiate into odontoblast-like cells, secrete dentin matrix, and promote revascularization within the pulp chamber.

In another example, when administered for periodontal regeneration, the pre-conditioned dental MSCs can be injected or placed within periodontal pockets, furcation defects, or gingival recessions. For example, the cells are suspended in a gel or paste formulation containing a scaffold such as chitosan, collagen, or fibrin, which allows adherence to the tooth root and surrounding connective tissue. The cell-laden matrix can be applied topically during open-flap debridement or guided tissue regeneration procedures to promote reattachment of periodontal ligament fibers and regeneration of alveolar bone and cementum.

In yet another example, for alveolar bone or maxillofacial bone regeneration, the pre-conditioned SHED cells can be incorporated into a solid or porous scaffold (e.g., PLGA, hydroxyapatite, or collagen sponge) and implanted directly into the bone defect, extraction socket, or implant site. The scaffold can be shaped to conform to the defect and secured with sutures or membranes to maintain space for new bone formation. The implanted cells contribute to osteogenesis and angiogenesis, accelerating bone regeneration and integration with surrounding tissue.

Following implantation of the pre-conditioned dental MSCs, standard post-implantation care and clinical management can be performed to ensure engraftment, vascularization, and tissue regeneration. For example, the method further includes sealing or stabilizing the implantation site using a suitable restorative or protective material, e.g., mineral trioxide aggregate (MTA), resin composite, collagen membrane, or fibrin sealant, to prevent microbial infiltration and maintain a sterile environment. The clinician (e.g., a dentist) can further apply a temporary or permanent dental restoration to protect the treated area from mechanical stress during early healing.

After implantation, the patient can be prescribed antibiotic prophylaxis and antiinflammatory or analgesic medication to reduce infection risk and discomfort. The treated area can be monitored for signs of inflammation, infection, or tissue rejection, and the patient is advised to maintain good oral hygiene and avoid excessive pressure or trauma to the treated tooth or site for a defined recovery period (e.g., 1-2 weeks).

In regenerative endodontic applications, follow-up evaluations can be performed periodically (e.g., 1, 3, and 6 months post-procedure) to assess tooth vitality, radiographic evidence of revascularization, and continued root development. For periodontal or bone regeneration, clinical probing depth, bone fill, and tissue attachment levels are evaluated over time.

In some forms, adjunctive therapies such as low-level laser therapy, platelet-rich plasma (PRP) application, or growth factor supplementation can be administered to enhance cell activation and tissue healing post-implantation. If the procedure involves a scaffold or hydrogel matrix, the degradation and integration of the material are monitored to confirm progressive replacement by newly formed tissue.

The disclosed compositions and methods can be further understood through the following numbered paragraphs:
Paragraph 1. Stem cells treated ex vivo with a compound in an amount effective to stabilize, activate, or express hypoxia-inducible factor-1 alpha (HIF-1α) in the stem cells under normoxic conditions.
Paragraph 2. The stem cells of paragraph 1, wherein the stem cells are obtained from human exfoliated deciduous teeth (SHED).
Paragraph 3. The stem cells of paragraph 1 or 2, wherein the compound is a Bcl-2 family inhibitor.
Paragraph 4. The stem cells of paragraph 3, wherein the Bcl-2 family inhibitor is selected from a group consisting of a small molecule, a peptide, an antibody, a nucleic acid, or a combination thereof.
Paragraph 5. The stem cells of paragraph 4, wherein the small molecule is TW-37, ABT-737, ABT-263, BM-1197, S44563, AZD4320, ABT-199, or S55746.
Paragraph 6. The stem cells of claim 4 or 5, wherein the small molecule is TW-37.
Paragraph 7. The stem cells of any one of paragraph 1-6, wherein the effective amount of the compound ranges from about 1 μM to 20 μM.
Paragraph 8. The stem cells of any one of paragraph 1-7, wherein the compound stabilizes, activates, or expresses HIF-1α through the modulation of adenylate kinase 4 (AK4).
Paragraph 9. The stem cells of paragraph 8, wherein the stabilization, activation, or expression of HIF-1α results in maintenance of ATP homeostasis.
Paragraph 10. The stem cells of paragraph 8, wherein the modulation of adenylate kinase 4 (AK4) maintains ATP homeostasis.
Paragraph 11. The stem cells of paragraph 8, wherein the compound results in maintenance of ATP homeostasis.
Paragraph 12. A method comprising administering the stem cells of any one of paragraph 1-11 to a subject.
Paragraph 13. The method of paragraph 12, wherein the treated stem cells enhance survival of the stem cells in the subject.
Paragraph 14. The method of paragraph 12 or 13, wherein the treated stem cells promote neovascularization in the subject.
Paragraph 15. The method of any one of paragraph 12-14, wherein the treated stem cells promote regeneration of vascularized tissue in the subject.
Paragraph 16. The method of any one of paragraph 12-15, wherein the stabilization or the activation of HIF-1α induces angiogenic and/or vasculogenic responses.
Paragraph 17. The method of paragraph 16, wherein the angiogenic and/or vasculogenic responses are mediated through the expression of an endothelial growth factor.
Paragraph 18. The method of paragraph 17, wherein the endothelial growth factor is vascular endothelial growth factor (VEGF), platelet-derived growth factor (PDGF), fibroblast growth factors (FGF), angiogenin, or epidermal growth factor (EGF).
Paragraph 19. The method of paragraph 17 or 18, wherein the endothelial growth factor is vascular endothelial growth factor (VEGF).
Paragraph 20. The method of any one of paragraph 12-19, wherein the stem cells are administered into the subject by intravenous (IV) injection, intramuscular (IM) injection, intra-articular injection, intrathecal injection, direct tissue injection, intra-arterial injection, subcutaneous injection, topical application, scaffold-based transplantation, or a combination thereof.
Paragraph 21. The method of any one of paragraph 12-20, wherein the administered stem cells promote neovascularization or vascular sprouting of endothelial cells through HIF-1α induced VEGF expression.
Paragraph 22. The method of any one of paragraph 12-21, wherein the stabilization, activation, or expression of HIF-1α under normoxia in SHED enhances therapeutic potential of the stem cells in SHED for dental pulp regeneration through augmentation of angiogenic properties and post-transplantation cell survival.

### EXAMPLES

### Materials and Methods

### Cells Culture and Small Molecules

SHED was purchased from AllCells, LLC (Alameda, CA, USA) and cultured in α-minimum essential medium (α-MEM) supplemented with 10% fetal bovine serum (FBS) and 1% penicillin/streptomycin (P/S) at 5% CO₂, 37° C. incubators. Human umbilical vein endothelial cells (HUVECs) were purchased from ScienCell (Carlsbad, CA, USA) and cultured in endothelial cell medium (ECM, ScienCell) supplemented with 5% FBS, 1% P/S, and 1% endothelial cell growth supplement at 5% CO₂, 37° C. incubators. TW-37 was commercially purchased from Beyotime (Shanghai, China). CoCl₂ and DFO were purchased from Sigma-Aldrich (USA).

### Western Blot

SHED cells were lysed by the RIPA buffer (Thermo) with 1% protease & phosphatase inhibitor cocktail (Thermo) after being rinsed with PBS. The BCA protein detection kit (Thermo) was used to detect the protein concentrations. The proteins were separated using the sodium dodecyl sulfate-polyacrylamide gel electrophoresis and transferred onto polyvinylidene difluoride membranes. The membranes were blocked with 5% milk in Tris-buffered saline containing Tween and then immunoblotted with primary antibodies. After that, the corresponding secondary antibodies were used to incubate the membranes. The membranes were visualized by the detection system (Thermo), and all the results were quantified by analyzing the integrated density with ImageJ software (National Institutes of Health), and the relative quantification of proteins was normalized to GAPDH.

### Cell Counting Kit-8 ((CK-8) Assay

Cell proliferation was detected using the CCK-8 reagents (APExBIO, USA) according to the instructions. Briefly, SHED were seeded into 96-well plates with 5×10³ cells and 100 μL medium per well, and after the treatment of TW-37, the cells were cultured for 24 h, 48 h, or 72 h, respectively. 10 μL of CCK-8 solution was added into each well of the plate, and the plate was incubated for 2 hours in the incubator. At the end, the absorbance was measured at 450 nm using a microplate reader (SpectraMax).

### Live and Dead Assay

A live/Dead Assay kit (ThermoFisher, USA) was used to detect the cell viability of TW-37 on SHED according to the manufacturer's protocol. After the vials were thawed, 5 μL calcein AM (Component A) and 20 μL ethidium homodimer-1 (Component B) were added to 10 mL PBS to create a staining solution. Then, remove the medium from the cells and add 100-200 μL of the staining solution directly to the cells. The cells were incubated for 30 minutes at room temperature, and images were captured by fluorescence microscope.

### RNA Sequencing and Analysis

SHED was treated with experimentally determined concentrations of the above-mentioned molecules or 1% oxygen for 3 h, and RNA was collected for profiling genome-wide gene expression changes using RNA-seq (Biomarker, China). The transcriptional profiles of genes in the microarray analyses were evaluated to identify upregulated and downregulated genes in all conditions.

### Enzyme-linked Immunosorbent Assay (ELISA)

Secretory VEGF levels in the culture supernatants were assessed by the Human VEGF DuoSet ELISA Kit (R & D Systems, Inc., USA) according to the protocol. Briefly, 96-well microplates were coated overnight with the capture antibody (diluted in PBS) and blocked for 1 hour with the Reagent Diluent. After washing with Wash Buffer (0.05% Tween 20 in PBS), 100 μL of samples or standards diluted in Reagent Diluent per well were added to the microplates and incubated at room temperature. After repeating the washing step, the Detection Antibody (diluted in Regent Diluent) was added to each well and incubated at room temperature. Then the working dilution of Streptavidin-HRP was added and incubated. Subsequently, the Substrate Solution was added and incubated for 20 minutes. Then, the Stop Solution was added to each well, and the plates were tapped gently for mixing. Finally, the absorbance density was determined under 450 nm and 540 nm by SpectraMax® microplate reader (Molecular Devices, CA, US). The readings at 540 nm were subtracted from those at 450 nm, and the concentrations of the samples were calculated by the standard curve. The results were expressed as pg/mL VEGF.

### In Vitro Matrigel Assay

In vitro Matrigel tube formation assay was conducted according to the manufacturer's (Corning® Matrigel® Matrix) protocol, using conditioned medium collected from SHED treated with TW-37, to assess the pro-angiogenic effects. Briefly, 10 μL of thawed Matrigel matrix was added to each well of the μ-Slide 15 well plate (Ibidi, Germany), and then incubated for 30 min at 37° C. to induce the gelation process. Subsequently, HUVEC cell suspension was seeded on to Matrigel coated plates and observed at regular intervals.

### Sprouting Assay

Sprouting assay was conducted using conditioned medium collected from SHED treated with TW-37 as described previously [26]. Images were taken using an invert microscope and cumulative sprouting length was measured with ImageJ software.

### Cell Transfection

The small interfering RNA (siRNA) of AK4 and the relevant negative control were designed by GenePharma. Totally 4 sequences of siRNA were generated, and western blot was done to determine the one with the highest transfection efficacy. The cell transfection was achieved by the Lipofectamine RNAiMAX reagents (Invitrogen) according to the instructions.

### Statistics

All data will be presented as mean±standard deviation and analyzed by Student's t-test or one-way analysis of variance (ANOVA) by using the GraphPad Prism software (San Diego, CA) with P<0.05 being considered statistically significant.

### Results

### TW-37 Induced HIF-1α Stabilization in SHED

As shown by western blot, the protein expression level of HIF-1α was significantly increased by TW-37 in SHED under normoxia in a dose-dependent manner at 6 h (FIGS. 1A-1B), 12 h (FIGS. 1C-1D) and 24 h (FIGS. 1E-1F), respectively.

### The Cell Viability of SHED was Inhibited by TW-37

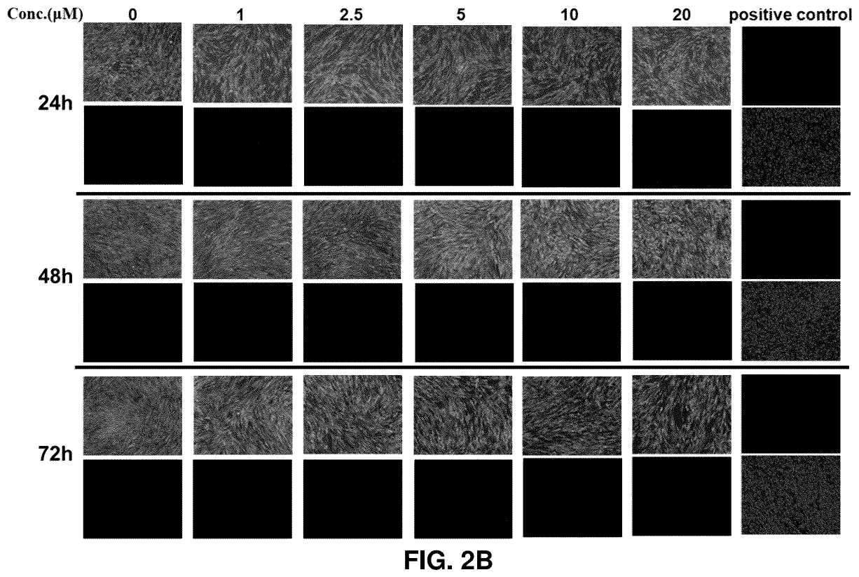

With different concentrations of TW-37 on SHED, the CCK-8 assay did not show statistical significance in cell proliferation at 24 h when the concentrations were less than 20 μM (FIG. 2A). But when the cells were cultured with TW-37 for 48 h and 72 h (FIG. 2A), there was a significant difference between the treated groups and the control group. From the results of the Live & Dead assay (FIG. 2B) almost no distinct dead cells were detected at 24 h when comparing the TW-37 groups with the control group, which indicated that TW-37 inhibited SHED proliferation but did not induce cell death.

### TW-37 Promoted the Secretion of VEGF in SHED

Secretory VEGF levels at 6 h (FIG. 3A), 12 h (FIG. 3B), and 24 h (FIG. 3C) in SHED with the treatment of TW-37 were increased markedly in a dose-dependent manner as detected by ELISA. Then, after the stimulation of TW-37 on SHED for 12 h or 24 h, the supernatant of the cells was removed, and fresh medium was added to the wells to continue culturing the cells for another 24 h. The ELISA results indicated that SHED could still secrete a high level of VEGF, significantly in 10 μM, and 20 μM groups, after TW-37 was removed (FIGS. 3D-3E). The above results indicated that TW-37 could directly and indirectly promote VEGF secretion.

### TW-37-Induced SHED Enhanced the Angiogenesis of HUVECs

To assess the influence of TW-37-induced SHED on angiogenesis, conditioned medium was collected after SHED was treated by TW-37 for 24 h. In vitro Matrigel tube formation assays showed that the numbers of nodes significantly increased at 6 h and 12 h, but with no significant increase after 24 h, which indicates that angiogenesis was enhanced at the early stage but that the tube formation would not be stabilized in the long term (FIGS. 4A-4B). The sprouting assay revealed that the conditioned medium from TW-37-induced SHED promoted the sprouting of HUVECs in a 3D spheroid model (FIG. 4C-4E). All these results revealed that TW-37-induced SHED enhanced the angiogenic property of HUVECs.

### TW-37 Did not Function as PHD Inhibitor in SHED

Western blot results showed that with the treatment of TW-37 on SHED, HIF-1α expression increased first and then decreased, during which the highest HIF-1α expression was at 3 h (FIG. 5A-5B). Interestingly, when HIF-1α was stabilized by TW-37, the expression of PHD was upregulated, which may be induced by the upregulation of HIF-1α (FIG. 5C-5E). Then, the function of TW-37 on Fe²⁺ was detected by ferrozine assay (FIG. 5F), which suggested that TW-37 could not act as a Fe²⁺ chelator in SHED.

TW-37 had a Strong Relationship with the HIF-1α Signaling Pathway in SHED by RNA Sequencing

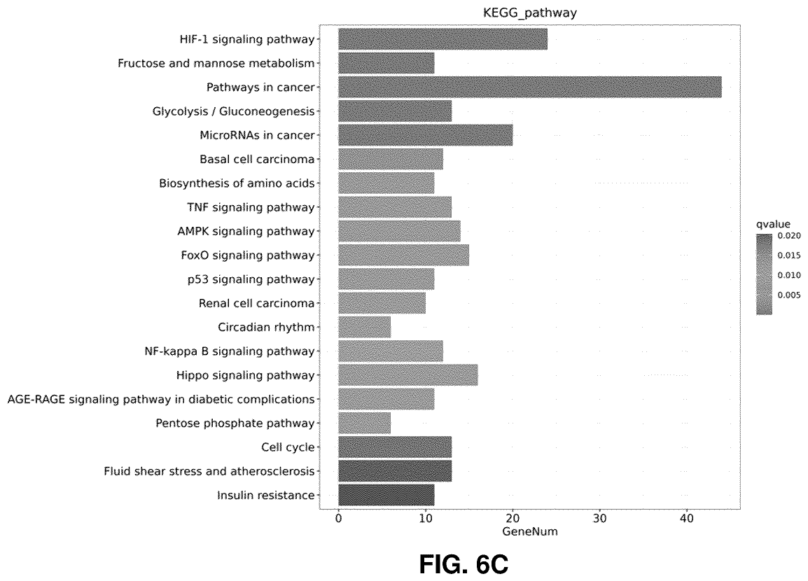

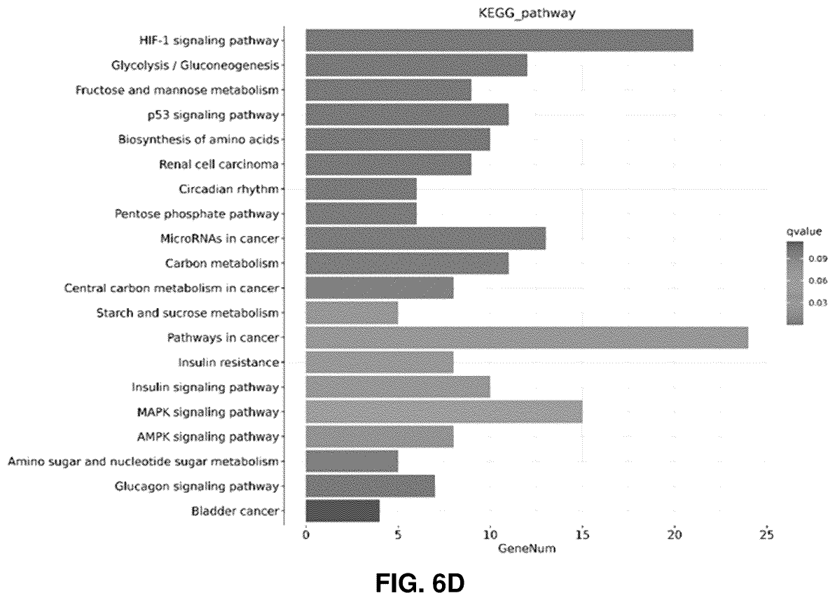

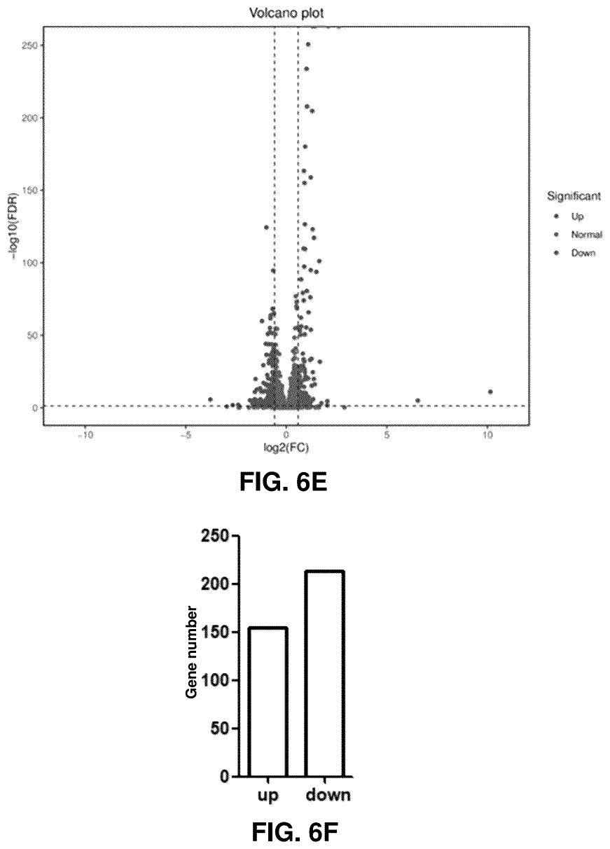

RNA sequencing was done to further explore the function and mechanism of TW-37 on SHED. The sequencing results demonstrated that there are 803 different expressed genes, including 479 upregulated genes and 324 downregulated genes at 3 h (FIG. 6A-6B). The analysis of the statistical pathway enrichment of all different expressed genes showed that the HIF-1α signaling pathway is the most involved pathway apart from the pathways in cancer (FIG. 6C). When it comes to the upregulated genes, it can be seen that the upregulated genes had a higher relation with the HIF-1α signaling pathway (FIG. 6D). Besides, genes that were differently expressed between the TW-37 group and the hypoxia group (the hypoxia chamber) were calculated to detect how TW-37 mimics hypoxia. The result showed that 367 different genes were expressed, including 154 upregulated genes and 213 downregulated genes (FIG. 6E-6F).

### TW-37 Enhanced HIF-1α Expression Through Regulating AK4

The results from the western blot showed that HIF-1α and AK4 (adenylate kinase 4) were both enhanced by TW-37 (FIG. 7A). Then, AK4 siRNA was used to further explore the interaction of HIF-1α and AK4. The protein level of AK4 was detected to confirm the efficiency of the siRNA, and si-2 was selected for the following experiments (FIG. 7B). The results showed that HIF-1α was inhibited when AK4 was knocked down with the treatment of TW-37 at 1.5 h (FIG. 7C) and 3 h (FIG. 7D). Taken together, these findings indicated that TW-37 induced HIF-1α stabilization through AK4.

Maintaining cell viability until adequate blood supply is achieved and inducing a timely angio-/vasculogenic response is important in cell-based tissue engineering strategies. The lentiviral-mediated gene knockdown methods are not clinically applicable due to associated safety concerns. TW-37, as a chemical compound, can be used to precondition SHED to hypoxia with HIF-1α stabilization ex vivo under normal oxygen conditions, which will make the dental stem cells more resistant to in vivo hostile environments and assist in achieving vascularized tissue regeneration.

TW-37, as a small molecule inhibitor of Bcl-2, is a previously unidentified product. In this application, it has been disclosed that the function of TW-37 is to stabilize HIF-1α through regulating adenylate kinase 4 (AK4) in SHED. The small molecule TW-37 can pretreat SHED in a desired dosage and induce the hypoxic preconditioning in SHED which permits HIF-1α expression in normoxia and in turn, enhances the secretion of VEGF.

TW-37 is a small molecule inhibitor of Bcl-2 designed using a structure-based design strategy. It has an anti-cancer role and functions as a potential apoptosis-inducing agent for the treatment of cancers. HIF-1α stabilization under normoxia achieved by TW-37 is an important approach to enhance the therapeutic potential of dental stem cells through augmentation of the angiogenic properties and post-implantation cell survival.

### REFERENCES

1. Cyrus C. Rabbani Michelle S. Hwang Patrick J. Byrne Shaun C. Desai. Management of large facial defects. Facial Plast Surg. 2020 April; 36(2):148-157.
2. Kao R T, Murakami S, Beirne O R. The use of biologic mediators and tissue engineering in dentistry. Periodontol 2000. 2009; 50:127-153.
3. Mas-Bargues C, Sanz-Ros J, Román-Domínguez A, Inglés M, Gimeno-Mallench L, El Alami M, Viña-Almunia J, Gambini J, Viña J, Borrás C. Relevance of oxygen concentration in stem cell culture for regenerative medicine. Int J Mol Sci. 2019 Mar. 8; 20(5):1195.
4. Haque N, Kasim N H, Rahman M T. Optimization of pre-transplantation conditions to enhance the efficacy of mesenchymal stem cells. Int J Biol Sci. 2015 Feb. 5; 11(3):324-334.
5. Shi, H. Hypoxia inducible factor 1 as a therapeutic target in ischemic stroke. Curr Med Chem. 2009; 16(34):4593-4600.
6. Lv B, Li F, Fang J, Xu L, Sun C, Han J, Hua T, Zhang Z, Feng Z, Jiang X. Hypoxia inducible factor 1α promotes survival of mesenchymal stem cells under hypoxia. Am J Transl Res. 2017 Mar. 15; 9(3):1521-1529.
7. Pan Z, Ma G, Kong L, Du G. Hypoxia-inducible factor-1: Regulatory mechanisms and drug development in stroke. Pharmacol Res. 2021 August; 170:105742.
8. Pugh C W, Ratcliffe P J. Regulation of angiogenesis by hypoxia: role of the HIF system. Nat Med. 2003 June; 9(6):677-684.
9. Hu C J, Iyer S, Sataur A, Covello K L, Chodosh L A, Simon M C. Differential regulation of the transcriptional activities of hypoxia-inducible factor 1 alpha (HIF-1αalpha) and HIF-2 alpha in stem cells. Mol Cell Biol. 2006; 26(9):3514-3526.
10. Linden, T. The antimycotic ciclopirox olamine induces HIF-1 & alpha; stability, VEGF expression, and angiogenesis. FASEB J. 2003 April; 17(6):761-763.
11. Miura M, Gronthos S, Zhao M, Lu B, Fisher L W, Robey P G, Shi S. SHED: stem cells from human exfoliated deciduous teeth. Proc Natl Acad Sci USA. 2003 May 13; 100(10):5807-5812.
12. Xu J G, Zhu S Y, Heng B C, Dissanayaka W L, Zhang C F. Tgf-beta1-induced differentiation of shed into functional smooth muscle cells. Stem Cell Res Ther. 2017 Jan. 23; 8(1):10.
13. Han Y, Gong T, Zhang C, Dissanayaka W L. HIF-1α stabilization enhances angio-/vasculogenic properties of SHED. J Dent Res. 2020 July; 99(7):804-812
14. Han Y, Koohi-Moghadam M, Chen Q, Zhang L, Chopra H, Zhang J, Dissanayaka W L. HIF-1α alpha stabilization boosts pulp regeneration by modulating cell metabolism. J Dent Res. 2022 September; 101(10):1214-1226.
15. Han Y, Chen Q, Zhang L, Dissanayaka W L. Indispensable role of HIF-1α signaling in post-implantation survival and angio-/vasculogenic properties of SHED. Front Cell Dev Biol. 2021 Jul. 23:9:655073.
16. Yoo H, Moon Y H, Kim M S. Effects of CoCl₂ on multi-lineage differentiation of C3h/10T1/2 mesenchymal stem cells. Korean J Physiol Pharmacol. 2016; 20(1):53-62.
17. Fujisawa K, Takami T, Okada S, Hara K, Matsumoto T, Yamamoto N, Yamasaki T, Sakaida I. Analysis of metabolomic changes in mesenchymal stem cells on treatment with desferrioxamine as a hypoxia mimetic compared with hypoxic conditions. Stem Cells. 2018 August; 36(8):1226-1236.
18. Liu G S, Peshavariya H M, Higuchi M, Chan E C, Dusting G J, Jiang F. Pharmacological priming of adipose-derived stem cells for paracrine VEGF production with deferoxamine. J Tissue Eng Regen Med. 2016; 10(3):E167-E176.
19. Zeng H L, Zhong Q, Qin Y L, Bu Q Q, Han X A, Jia H T, Liu H W. Hypoxia-mimetic agents inhibit proliferation and alter the morphology of human umbilical cord-derived mesenchymal stem cells. BMC Cell Biol. 2011 Aug. 9; 12:32.
20. Yang I H, Ahn C H, Cho N P, Jin B, Lee W, Jung Y C, Hong S D, Shin J A, Cho S D. Heme Oxygenase-1 is a key molecule underlying differential response of TW-37-induced apoptosis in human mucoepidermoid carcinoma cells. Molecules. 2019 May 1; 24(9):1700.
21. Ahn C H, Lee W W, Jung Y C, Shin J A, Hong K O, Choi S, Swarup N, Kim J, Ahn M H, Jung M, Cho S D, Jin B. Antitumor effect of TW-37, a BH3 mimetic in human oral cancer. Lab Anim Res. 2019; 35:194-201.
22. Yang I H, Hong S H, Jung M, Ahn C H, Yoon H J, Hong S D, Cho S D, Shin J A. Cryptotanshinone chemosensitivity potentiation by TW-37 in human oral cancer cell lines by targeting STAT3-Mcl-1 signaling. Cancer Cell Int. 2020 Aug. 26; 20:405.
23. Jiang L, Peng W W, Li L F, Du R, Wu T T, Zhou Z J, Zhao J J, Yang Y, Qu D L, Zhu Y Q. Effects of deferoxamine on the repair ability of dental pulp cells in vitro. J Endod. 2014 August; 40(8):1100-1104.
24. Laksana K, Sooampon S, Pavasant P, Sriarj W. Cobalt chloride enhances the stemness of human dental pulp cells. J Endod. 2017 May; 43(5):760-765.
25. Osathanon T, Vivatbutsiri P, Sukarawan W, Sriarj W, Pavasant P, Sooampon S. Cobalt chloride supplementation induces stem-cell marker expression and inhibits osteoblastic differentiation in human periodontal ligament cells. Arch Oral Biol. 2015 January; 60(1):29-36.
26. Zhang Y, Liu J, Zou T, Qi Y, Yi B, Dissanayaka W L, Zhang C. DPSCs treated by TGF-beta1 regulate angiogenic sprouting of three-dimensionally co-cultured HUVECs and DPSCs through VEGF-Ang-Tie2 signaling. Stem Cell Res Ther. 2021 May 10; 12(1):281.

It is understood that the disclosed method and compositions are not limited to the particular methodology, protocols, and reagents described as these can vary. It is also to be understood that the terminology used herein is for the purpose of describing particular embodiments only and is not intended to limit the scope of the present invention which will be limited only by the appended claims.

Those skilled in the art will recognize or be able to ascertain using no more than routine experimentation many equivalents to the specific embodiments of the method and compositions described herein. Such equivalents are intended to be encompassed by the following claims.

We claim:

1. Stem cells treated ex vivo with a pre-conditioning agent in an amount effective to stabilize, activate, or express hypoxia-inducible factor-1 alpha (HIF-1α) in the stem cells under normoxic conditions.

2. The stem cells of claim 1, wherein the dental stem cells are obtained from human exfoliated deciduous teeth (SHED).

3. The stem cells of claim 1, wherein the pre-conditioning agent is a Bcl-2 family inhibitor selected from a group consisting of TW-37, ABT-737, ABT-263 (Navitoclax), BM-1197, S44563, AZD4320, ABT-199 (Venetoclax), and S55746, or a pharmaceutically acceptable salt, solvate, or derivative thereof.

4. The stem cells of claim 3, wherein the Bcl-2 family inhibitor is TW-37.

5. The stem cells of claim 1, wherein the effective amount of the pre-conditioning agent ranges from about 1 μM to 20 μM.

6. The stem cells of claim 1, wherein the pre-conditioning agent increases the expression of HIF-1α, adenylate kinase 4 (AK4), VEGF, or a combination thereof in the stem cells.

7. The stem cells of claim 1, further comprising a pharmaceutically acceptable carrier or excipient.

8. A method comprising promoting dental or craniofacial tissue regeneration in a subject in need thereof, comprising:
(a) administering an effective amount of the pre-conditioned stem cells of claim 1 to a target site of the subject,
wherein the pre-conditioned stem cells are effective to increase angiogenesis, stem cell survival, tissue regeneration, or a combination thereof, at the target site relative to a control.

9. The method of claim 8, wherein the pre-conditioned stem cells increase neovascularization at the target site in the subject relative to a control.

10. The method of claim 8, wherein the pre-conditioned stem cells increase regeneration of vascularized tissue in the subject.

11. The method of claim 8, wherein the increased angiogenesis comprises increased expression of an endothelial growth factor selected from the group consisting of vascular endothelial growth factor (VEGF), platelet-derived growth factor (PDGF), fibroblast growth factors (FGF), angiogenin, or epidermal growth factor (EGF).

12. The method of claim 8, wherein the pre-conditioned stem cells are administered to the subject as an injectable cell suspension, a hydrogel or paste formulation, or a scaffold-based implant comprising the pre-conditioned stem cells in a biocompatible matrix,
wherein the biocompatible matrix comprises collagen, fibrin, alginate, hyaluronic acid, chitosan, gelatin, polylactic acid (PLA), polyglycolic acid (PGA), or a copolymer thereof.

13. The method of claim 8, wherein the target site is selected from a dental pulp chamber, root canal space, periapical lesion, periodontal defect, extraction socket, alveolar bone defect, or maxillofacial soft-tissue defect, or a combination thereof.

14. The method of claim 8, wherein the administering comprises injecting the pre-conditioned stem cells into the target site or applying pre-conditioned stem cells via topical administration.

15. The method of claim 8, further comprising, after step (a), sealing the target site with a restorative material to prevent contamination of the target site.

16. The method of claim 8, wherein the pre-conditioned stem cells are administered in an amount between 1×10⁴ to 1×10⁷ viable cells.

17. The method of claim 8, wherein the stem cells are administered into the subject by intravenous (IV) injection, intramuscular (IM) injection, intra-articular injection, intrathecal injection, direct tissue injection, intra-arterial injection, subcutaneous injection, topical application, scaffold-based transplantation, or a combination thereof.
